(19) United States
(12) Patent Application Publication
(10) Pub. No.: US 2025/0144042 A1
(43) Pub. Date: May 8, 2025

(54) COMPOSITIONS AND METHODS TO ACHIEVE SYSTEMIC UPTAKE OF PARTICLES FOLLOWING ORAL OR MUCOSAL ADMINISTRATION

(71) Applicant: Brown University, Providence, RI (US)

(72) Inventors: Edith Mathiowitz, Providence, RI (US); Aharon Azagury, Providence, RI (US); Kosta Milovanovic, Providence, RI (US); Cameron Baptista, Providence, RI (US); Carder Jones, Providence, RI (US); Eric Darling, Providence, RI (US)

(21) Appl. No.: 18/840,305

(22) PCT Filed: Feb. 22, 2023
(86) PCT No.: PCT/US2023/063041

§ 371 Date: Aug. 21, 2024

Related U.S. Application Data
(60) Provisional application No. 63/312,672, filed on Feb. 22, 2022.

(51) Int. Cl. A61K 9/51

(52) U.S. Cl. CPC A61K 9/5161; A61K 9/5123; A61K 9/5169

(57) ABSTRACT

Described are coated particles containing a core and a coating, and pharmaceutical formulations containing these coated particles. The core contains a polymer or a hydrophobic drug. The coating contains a glycoprotein or a combination of a sugar and a protein. The coating surrounds the core. The coated particles are effectively absorbed by mucosa such as intestinal mucosa, GI tissue, and/or vaginal mucosa, and show increased systemic uptake following oral or mucosal administration. Optionally, the coated particles contain one or more active agents encapsulated in the core and/or embedded in the coating of the particles, for systemic or local delivery.

### CROSS-REFERENCE TO RELATED APPLICATION

This application claims benefit and priority to U.S. Application No. 63/312,672, filed Feb. 22, 2022, the disclosure of which is incorporated herein by reference.

### FIELD OF THE INVENTION

This invention is generally in the area of compositions for delivery of therapeutic, diagnostic, and/or prophylactic agents, particularly the delivery of these agents by oral and/or mucosal administration.

### BACKGROUND OF THE INVENTION

Oral drug delivery is a preferred route for drug administration. Substantial effort has been dedicated to the development of oral delivery systems based on polymeric particles. The use of oral delivery has been limited by stomach acidity, enzymatic activity, and low penetration through the GI tissue. Most of the introduced polymeric particles are entrapped and eliminated by the protective mucosal lining of the GI tract, significantly reducing the efficiency of such delivery systems.

The mucosal membrane, considered the main barrier for mass transport, is a mucoadhesive layer made of hydrophilic and hydrophobic regions containing mainly glycoproteins (e.g. mucins) (Sarosiek, et al. Biochem. Biophys. Res. Commun. 118, 523-531 (1984); Witas, et al. Carbohydr. Res. 120, 67-76 (1983); Lichtenberger, Annu. Rev. Physiol. 57, 565-583 (1995)). It contains two layers: a firmly adherent lower layer and a loose upper layer adjacent to the epithelial lining with a mesh structure of 200-500 nm pores (Cu & Saltzman, Adv. Drug Deliv. Rev. 61, 101-114 (2009); Jung, et al. European Journal of Pharmaceutics and Biopharmaceutics 50, 147-160 (2000)). The upper layer is loosely adherent and much less viscous. In order to overcome oral delivery barriers, different nanoparticle vehicles have been utilized (Ensign, et al. Adv. Drug Deliv. Rev. 64, 557-570 (2012); Kumari, et al. Colloids and Surfaces B: Biointerfaces 75, 1-18 (2010); Panyam & Labhasetwar, Adv. Drug Deliv. Rev. 55, 329-347 (2003); Plapied, et al. Current opinion in colloid & interface science 16, 228-237 (2011); Reineke, et al. J. Controlled Release 170, 477-484 (2013); Reineke, et al. Proc.Natl.Acad.Sci. U.S.A. 110, 13803-13808 (2013)).

The mucus lining of GI tract successfully entraps and eliminates the majority of encapsulating materials, making it hard to achieve effective amounts of these agents systemically or in some cases hard to achieve effective amounts of the agents in the intestinal epithelial tissue (i.e., local delivery). Some studies have observed bioadhesive nanoparticles being entrapped in mucus then cleared (Lai, et al. Proc. Natl. Acad. Sci. U.S.A. 104, 1482-1487 (2007); des Rieux, et al. J. Controlled Release 116, 1-27 (2006)). US 2021/0186880 by Mathiowitz, et al. discloses bioadhesive polymeric particles with negative zeta potentials being absorbed by intestinal mucosa, and speculates that mucin has the ability to coat these particles and thereby reduce the negative charge on the particles. However, such particles suffer from aggregation in the GI mucosa in vivo and thereby limit their delivery efficiency.

There remains a need for improved compositions for delivery of active agents by oral and/or mucosal administration.

Therefore, it is an object of the invention to provide improved particles for oral and/or mucosal drug delivery.

It is a further objection of the invention to provide compositions for delivery of active agent(s) by oral and/or mucosal administration.

It is another object of the invention to provide methods for increasing uptake or bioavailability of active agent(s) following oral and/or mucosal administration.

It is another object of the invention to provide improved methods for treating, preventing, and/or ameliorating one or more symptoms associated with a disease or disorder in a subject.

### SUMMARY OF THE INVENTION

Described are coated particles, pharmaceutical formulations containing these coated particles, methods for treating or preventing a disease or disorder or a symptom associated therewith in a subject, and methods for ameliorating one or more symptoms associated with a disease or disorder in a subject. The coated particles contain a core and a coating. The core can be a polymeric core or a hydrophobic drug. The coating contains a glycoprotein (e.g. mucin, Glycoprotein-41, Glycoprotein-120, etc.) or a combination of a sugar, an oligosaccharide, and/or a polysaccharide and a protein that contains an amino acid backbone containing at least about 50 repeated units of serine and/or threonine. For example, the amino acid backbone contains repeated units of serine and/or threonine and the total number of the serine and/or threonine is at least 50. The coating of the particle is formed prior to administration of the particle to a mucosal surface of a subject and is not a biocoating. Biocoating refers to a spontaneous coating that is formed in vivo. The glycoprotein or the combination of sugar, oligosaccharide, and/or polysaccharide and protein surround the core. When the combination of a sugar (e.g. sucrose, glucose, lactose, etc.), oligosaccharide (e.g. oligomer of sucrose, glucose, lactose, etc.), and/or polysaccharide (e.g. polymer of sucrose, glucose, lactose, etc.), and a protein form the coating of the particle, the sugar, oligosaccharide, and/or polysaccharide can be covalently attached to or non-covalently associated with the amino acid backbone of the protein.

The coated particles and pharmaceutical formulations described herein have improved properties for drug delivery via oral and/or mucosal administration, such as increased diffusion coefficient and/or reduced particle aggregation in mucus, increased systemic uptake into blood, and/or increased local GI uptake. The coated particles are absorbed by a mucous membrane, such as intestinal mucosa and/or tissue. The coated particles are expected to increase systemic uptake following oral and/or mucosal administration, compared to a control particle containing the same polymeric core or hydrophobic drug without a coating.

In some forms, the core of the coated particle is a polymeric core. Coated particles containing polymeric cores are referred to herein as “coated polymeric particles.” Polymers suitable for forming the polymeric core are generally biodegradable and biocompatible polymers; optionally the polymers have a molecular weight between 1.5 kDa and 300 kDa. Optionally, the core of the coated polymeric particle contains a bioadhesive polymer, optionally the polymer, when measured in bulk, having a bioadhesion force of 100 mN/cm² or greater. In some forms, the core of the coated particle is formed by a hydrophobic drug, optionally more than one hydrophobic drug. The hydrophobic drug or each hydrophobic drug of two or more drugs forming the core can be a single molecule of the drug or multiple molecules of the drug. Hydrophobic drugs suitable for forming the core are generally compounds that lack affinity for or repelling water, such as BCS Class IV drugs.

Coated polymeric particles can also contain one or more active agents. The active agents may be therapeutic agents, prophylactic agents, and/or diagnostic agents. The active agents can be encapsulated in the polymeric core and/or embedded in the coating of the coated particles. Coated particles that contain one or more hydrophobic drugs as the core, can also contain one or more different active agents in the coating of the coated particles.

In some forms, the coated particles have a diffusion coefficient in mucus or a mucin solution (e.g. 3%-7% w/v mucin, such as 5% w/v mucin) that is at least 1.1-fold, at least 1.3-fold, at least 2-fold, at least 5-fold, at least 10-fold, at least 15-fold, at least 20-fold, in a range from 1.3-fold to 50-fold, from 1.3-fold to 40-fold, from 1.3-fold to 30-fold, from 1.3-fold to 25-fold, from 2-fold to 50-fold, from 2-fold to 40-fold, from 2-fold to 30-fold, from 2-fold to 25-fold, from 5-fold to 50-fold, from 5-fold to 40-fold, from 5-fold to 30-fold, or from 5-fold to 25-fold greater than the diffusion coefficient of control particles containing the core only in the same mucus or mucin solution, under the same measurement conditions. Optionally, the coated particles show a reduced particle aggregation in mucus or a mucin solution (e.g. 0.1% or 5% w/v mucin), as shown by a reduced number of clusters detected, by at least 5%, at least 10%, in a range from 5% to 50%, from 5% to 40%, from 10% to 50%, or from 20% to 50%, compared to the number of clusters detected for control particles in the same mucus or mucin solution, under the same measurement conditions.

In some forms, the coated particles have a diffusion coefficient in mucus or a mucin solution (e.g. 3%-7% mucin, such as 5% mucin) that is at least 1.1-fold, at least 1.3-fold, at least 2-fold, at least 5-fold, at least 10-fold, at least 20-fold, at least 30-fold, at least 40-fold, at least 50-fold, at least 100-fold, at least 150-fold, at least 160-fold, at least 170-fold, at least 200-fold, at least 250-fold, at least 300-fold, at least 500-fold, at least 1000-fold, at least 5000-fold, in a range from 1.3-fold to 10,000-fold, from 1.3-fold to 8000-fold, from 1.3-fold to 5000-fold, from 1.3-fold to 1000-fold, from 1.3-fold to 500-fold, from 1.3-fold to 200-fold, from 1.3-fold to 100-fold, from 1.3-fold to 30-fold, from 1.3-fold to 25-fold, from 2-fold to 30-fold, from 2-fold to 25-fold, from 5-fold to 30-fold, from 5-fold to 25-fold, from 100-fold to 10,000-fold, from 100-fold to 8000-fold, from 100-fold to 5000-fold, from 100-fold to 1000-fold, from 100-fold to 500-fold, from 150-fold to 10,000-fold, from 150-fold to 8000-fold, from 150-fold to 5000-fold, from 150-fold to 1000-fold, from 150-fold to 500-fold, or from 500-fold to 10,000-fold, greater than the diffusion coefficient of control particles containing the core only in water, under the same measurement conditions.

In some forms, the control particles containing the core only have a diffusion coefficient in mucus or a mucin solution (e.g. 3%-7% mucin, such as 5% mucin) that is at least 1.1-fold, at least 1.3-fold, at least 2-fold, at least 5-fold, at least 10-fold, at least 20-fold, at least 30-fold, at least 40-fold, at least 50-fold, at least 100-fold, at least 150-fold, at least 160-fold, at least 170-fold, at least 200-fold, at least 250-fold, at least 300-fold, in a range from 1.3-fold to 500-fold, from 1.3-fold to 400-fold, from 1.3-fold to 300-fold, from 1.3-fold to 200-fold, greater than the diffusion coefficient of the control particles in water, under the same measurement conditions.

In some forms, the diffusion coefficient of the coated particles in mucus or a mucin solution (e.g. 3%-7% mucin, such as 5% mucin) has a first enhancement factor, compared to the diffusion coefficient of control particles containing the core only in water, under the same measurement conditions; the diffusion coefficient of the same control particles in mucus or a mucin solution (e.g. 3%-7% mucin, such as 5% mucin) has a second enhancement factor, compared to the diffusion coefficient of the same control particles in water, under the same measurement conditions. In these forms, the first enhancement factor is at least 1.1-fold, at least 1.5-fold, at least 2-fold, at least 3-fold, at least 5-fold, at least 6-fold, at least 7-fold, at least 8-fold, at least 10-fold, at least 12-fold, at least 15-fold, at least 20 fold, in a range from 1.1-fold to 100-fold, from 1.1-fold to 80-fold, from 1.1-fold to 60-fold, from 1.1-fold to 50-fold, from 1.1-fold to 40-fold, from 1.1-fold to 30-fold, from 2-fold to 100-fold, from 2-fold to 80-fold, from 2-fold to 60-fold, from 2-fold to 50-fold, from 2-fold to 40-fold, from 2-fold to 30-fold, from 5-fold to 100-fold, from 5-fold to 80-fold, from 5-fold to 60-fold, from 5-fold to 50-fold, from 5-fold to 40-fold, or from 5-fold to 30-fold, such as about 1.1-fold, about 5-fold, or about 20-fold, greater than the second enhancement factor.

In some forms, when the coated particles are administered to a mammal, such as by oral administration or mucosal administration (local), systemic uptake of the coated particles is in a range from 10% to 90%, from 15% to 85%, from 15% to 80%, from 15% to 70%, from 15% to 60%, from 15% to 50%, from 20% to 90%, from 20% to 80%, from 20% to 70%, from 20% to 60%, or from 20% to 50% in the mammal, such as about 30%, as measured using Fourier Transform Infrared spectroscopy.

Also described are methods of making and using the coated particles. Such methods include methods for treating or preventing a disease or disorder or a symptom associated therewith in a subject, and methods for ameliorating one or more symptoms associated with a disease or disorder in a subject.

### BRIEF DESCRIPTION OF THE DRAWINGS

FIG. 1 is a bar graph showing the hydrodynamic diameter (volumetric average) of particles before and after coating with mucin.

FIGS. 2A-2H are graphs showing detected clusters of PLA8, PCRPS, PS 125-250, and PMMA NPs in 5% mucin solution. FIGS. 2A-2D show the clusters detected when the PMMA (FIG. 2A), PLA8 (FIG. 2B), PS 125-250 (FIG. 2C), and PCRPS (FIG. 2D) NPs were first dispersed in DI water. FIGS. 2E-2H show the clusters detected when the PMMA (FIG. 2E), PLA8 (FIG. 2F), PS 125-250 (FIG. 2G), and PCRPS (FIG. 2H) NPs were first dispersed in 0.1% mucin solution.

FIG. 3 is a three-dimensional bar graph showing the diffusion coefficients and population distributions of PS NPs having an average diameter of about 0.5 micron in DI water (“PS Control”), in 5% w/v mucin without initial dispersion in mucin (“Control in Mucin”), in 5% w/v mucin with initial dispersion in 0.1% w/v mucin (“PS 0.1 Coat in Mucin”), or in 5% w/v mucin with initial dispersion in 1.0% w/v mucin (“PS 1.0 Coat in Mucin”).

FIG. 4 is a three-dimensional bar graph showing the diffusion coefficients and population distributions of PS NPs having an average diameter of about 1.0 micron in DI water (“PS Control”), in 5% w/v mucin without initial dispersion in mucin (“Control in mucin”), in 5% w/v mucin with initial dispersion in 0.1% w/v mucin (“PS 0.1 Coat in Mucin”), or in 5% w/v mucin with initial dispersion in 1.0% w/v mucin (“PS 1.0 Coat in Mucin”).

### DETAILED DESCRIPTION OF THE INVENTION

### I. Compositions

Coated particles and formulations containing these coated particles for oral and/or mucosal delivery are described herein. The coated particles described herein generally demonstrate enhanced diffusion in mucosa (e.g. intestinal and intravaginal mucosa and/or tissue) and/or reduced aggregation. The coated particles are expected to increase systemic uptake following oral and/or mucosal administration compared to control (uncoated) particles.

### A. Coated Particles

The coated particle contains a core and a coating. The core can be a polymeric core or a hydrophobic drug. The coating contains a glycoprotein (e.g. mucin, Glycoprotein-41, Glycoprotein-120, etc.) or a combination of a sugar, an oligosaccharide, and/or a polysaccharide, and a protein that contains an amino acid backbone containing at least about 50 repeated units of serine and/or threonine. For example, the amino acid backbone contains repeated units of serine and/or threonine and the total number of the serine and/or threonine is at least 50. The coating of the particle is formed on the core prior to administration of the particle to a mucosal surface of a subject, and is not a biocoating that is spontaneously formed in vivo following administration. The glycoprotein or the combination of sugar, oligosaccharide, and/or polysaccharide and protein surrounds the core. When the combination of a sugar (e.g. sucrose, glucose, lactose, etc.), oligosaccharide (e.g. oligomer of sucrose, glucose, lactose, etc.), and/or polysaccharide (e.g. polymer of sucrose, glucose, lactose, etc.), and a protein form the coating of the particle, the sugar, oligosaccharide, and/or polysaccharide can be covalently attached to or non-covalently associated with the amino acid backbone of the protein.

When the coated particles contain polymeric cores, the coated particles contain one or more active agent(s), such as one or more therapeutic agents, one or more prophylactic agents, and/or one or more diagnostic agents. The active agent(s) can be encapsulated in the polymeric core or embedded in the coating of the coated particles, or a combination thereof. When the coated particles contain hydrophobic drug(s) as the core, one or more active agents can optionally be embedded in the coating of the coated particles.

The coated particles disclosed herein can have a diffusion coefficient in mucus or a mucin solution (e.g. 3%-7% mucin, such as 5% mucin) that is greater than the diffusion coefficient of control particles in the same mucus or mucin solution, under the same measurement conditions. As used herein the term “control particles” refers to particles that contain the same core as the coated particles but without the coating. For example, for coated particles containing a polymeric core and coating, the control particles contain the same polymeric core without the coating. For coated particles containing a hydrophobic drug core with a coating, the control particles contain the same hydrophobic drug core without the coating. The term “same measurement conditions” refers to measurement at the same temperature, pressure, time period, observation window, etc. Optionally, the coated particles also show a reduced particle aggregation in mucus or a mucin solution (e.g. 3%-7% mucin, such as 5% mucin), as shown by a reduced number of clusters detected, compared to the number of clusters detected for control particles in the same mucus or mucin solution, under the same measurement conditions. A “cluster” refers to an aggregate of four or more coated particles or control particles.

In some forms, the coated particles have a diffusion coefficient in mucus or a mucin solution (e.g. 3%-7% mucin, such as 5% mucin) that is greater than the diffusion coefficient of control particles containing the core only in water, under the same measurement conditions.

In some forms, the control particles containing the core only have a diffusion coefficient in mucus or a mucin solution (e.g. 3%-7% mucin, such as 5% mucin) that is greater than the diffusion coefficient of the control particles in water, under the same measurement conditions.

In some forms, the diffusion coefficient of the coated particles in mucus or a mucin solution (e.g. 3%-7% mucin, such as 5% mucin) has a first enhancement factor, compared to the diffusion coefficient of control particles containing the core only in water; the diffusion coefficient of the same control particles in mucus or a mucin solution (e.g. 3%-7% mucin, such as 5% mucin) has a second enhancement factor, compared to the diffusion coefficient of the same control particles in water. In these forms, the first enhancement factor is greater than the second enhancement factor. The term “enhancement factor” refers to the fold increase of diffusion coefficient of the coated particles or control particles in mucus or a mucin solution compared to the diffusion coefficient of the same coated particles or control particles in water, under the same measurement conditions.

When the coated particles are administered to a mammal, such as by oral administration or mucosal administration (such as local administration to a mucosal surface), the systemic uptake of the coated particles can reach an appreciable level, such as in a range from 10% to 90%, for example, about 30% in the blood of the mammal, as determined using Fourier Transform Infrared spectroscopy (“FTIR”).

### 1. Core

The core of the coated particle can be a polymeric core or a hydrophobic drug.

a. Polymeric Core

In some forms, the coated particle contains a polymeric core. The polymeric core contains a polymer (e.g. homopolymer or copolymer) or a blend of two or more polymers. When the core of the coated polymeric particle contains a blend of polymers, the polymers can be the same type of different types. For example, the core of the coated polymeric particle contains a blend of two polymers, where the two polymers are the same type but have different molecular weights (e.g. a blend of polystyrenes having different molecular weights). For example, the core of the coated particles contains a blend of two or more polymers, where the polymers are the same type but have a range of molecular weights, such as PS 125-250 KDa that contains a blend of polystyrene polymers having molecular weights in a range from 125 Kda to 250 Kda. For example, the core of the coated polymeric particle contains a blend of two polymers, where a first polymer is a type that is different from a second polymer (e.g. a blend of poly(lactic acid) and poly(glycolic acid)). However, the core is not formed from pegylated polymers, i.e. a polymer to which polyethylene glycol (PEG) is covalently and non-covalently attached. Further, the core is not pegylated, i.e. PEG is not covalently or non-covalently attached to or forming the outer surface of the core.

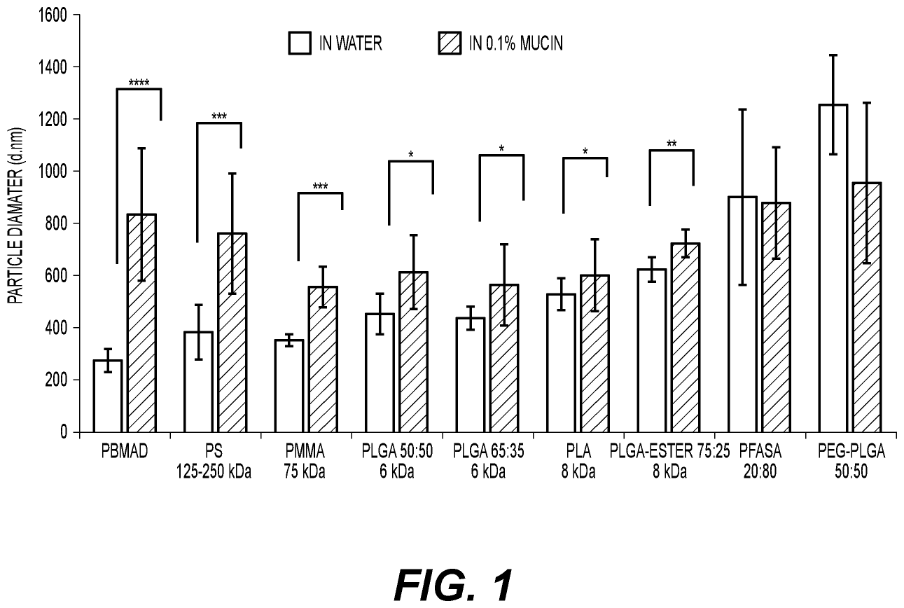

The polymer(s) forming the core of the coated particles is/are neutral or negatively or positively charged or contain(s) one or more moieties (i.e. one or more functional groups) that impart a negative or positive charge to the polymers in water. Optionally, these moieties are present on the surface of the core, such that the core of the coated particles itself displays negative or positive zeta potentials in DI water. For example, the core of the coated polymeric particle itself has a negative zeta potential in a range from −15 mV and −80 mV, such as from −20 mV to −70 mV, in DI water at room temperature. For example, the core of the coated polymeric particle itself has a positive zeta potential in a range from +15 mV and +80 mV, such as from +20 mV to +70 mV, in DI water at room temperature. When the core is coated, the coating surrounding the polymeric core can affect the negative or positive charge of the polymeric core such that the coated particle has a near neutral zeta potential, measured in deionized water at room temperature using a Zetasizer or similar instrument such as Zetaview. The term “room temperature” refers to a temperature in a range from 288 K to 303 K, such as about 298 K. The term “near neutral zeta potential” refers to a zeta potential in a range from −16 mV to +16 mV. Such a change of zeta potential is a way of demonstrating that a coating surrounds a previously charged polymeric core. Similarly, measuring the hydrodynamic diameter of a core before and after coating, and detecting an increase in the hydrodynamic diameter after applying a coating, such as a mucin coating, demonstrates that the coating formed around the core (see, e.g., FIG. 1).

i. Polymers

Suitable polymers for forming the core of the coated particles are biodegradable and biocompatible polymers that degrade rather than dissolve in an ageuous medium. Polymer dissolution can be determined as described in Estrellas, et al., Colloids and Surfaces B: Biointerfaces 173 (2019), 454-469, the contents of which are hereby incorporated by reference. In some forms, polymer dissolution can be determined as a function of time, i.e., rate of dissolution, at a given pH. For example, polymers can remain intact for a certain time period (e.g. about one hour) and pH (e.g. between 6 and 7), and subsequently dissolve. These biodegradable and biocompatible polymers can be homopolymers, copolymers, or a combination thereof.

Biodegradable and biocompatible polymers that are suitable for forming the core of the coated particles can be hydrophobic, hydrophilic, or amphiphilic, selected based on the specific application, such as the specific active agents for delivery and the administration route. Whether a polymer is hydrophobic or hydrophilic can also be determined via contact angle. For example, if a polymer is applied to a surface, such as glass, and forms a contact angle with water, which is greater than the contact angle of water on a surface of glass without the polymer, the polymer is hydrophobic. If a polymer is applied to a surface, such as glass, and forms a contact angle with water, which is smaller than the contact angle of water on a surface of glass without the polymer, the polymer is hydrophilic.

When a copolymer is used to form the core of the coated particle, the weight percentage of each monomer in the copolymer can vary from 5% to 95%. For example, a copolymer formed by two different monomers is used to form the core of the coated particle, where the weight percentage of a first monomer or a second monomer is in a range from 5% to 95%, such as from 5% to 90%, from 5% to 80%, from 5% to 75%, from 10% to 90%, from 10% to 75%, from 20% to 90%, from 20% to 80%, from 25% to 75%, from 25% to 50%, from 30% to 90%, from 40% to 90%, from 50% to 90%, or from 50% to 75%.

When a blend of polymers is used to form the core of the coated particle, the weight percentage of each polymer in the blend can vary from 5% to 95%. For example, a blend of two polymers is used to form the core of the coated particle, where the weight percentage of a first polymer or a second polymer is in a range from 5% to 95%, such as from 5% to 90%, from 5% to 80%, from 5% to 75%, from 10% to 90%, from 10% to 75%, from 20% to 90%, from 20% to 80%, from 25% to 75%, from 25% to 50%, from 30% to 90%, from 40% to 90%, from 50% to 90%, or from 50% to 75%.

Generally, any biodegradable and biocompatible polymers, regardless of their composition (i.e. homo- or poly-) and hydrophilicity, can be used for forming the core of the coated particles.

ii. Exemplary Polymers

Examples of biodegradable and biocompatible polymers that are suitable for forming the core of the coated particle include, but are not limited to, hydrogels, such as fibrin, collagen, gelatin, hyaluronic acid, alginate, cellulose, dextran, and agarose, poly(alkylene glycol), such as poly(ethylene glycol), poly(vinyl alcohol), poly(acrylic acid), polyacrylate, such as poly(methyl methacrylate), poly(2-hydroxyethyl methacrylate), poly(ethyleneglycolmethacrylate), poly(oligoethylene glycol methacrylate), poly(ethyleneglycol dimethacrylate), and poly(diacrylate), polyacrylamide, such as poly(isopropylacrylamide), poly(vinyl pyrrolidone), hydrophobic peptides, polyesters, such as poly(caprolactone), poly(hydroxyacids), such as poly(lactic acid), poly(glycolic acid), and poly(lactic acid-co-glycolic acid), polyhydroxyalkanoates, such as poly(3-hydroxybutyrate) and poly(4-hydroxybutyrate), polyanhydrides, such as poly(fumaric-co-sebacic acid), poly[butadiene-co-(maleic anhydride)], poly(butadiene-maleic anhydride-co-L-DOPA), polysebacic acid, and polyfumaric acid, poly(orthoesters), hydrophobic polypeptides, hydrophobic polyethers, such as poly(propylene oxide), poly(phosphazenes), polyesteramides, poly(alkylene alkylates), polyether esters, polyacetals, polycyanoacrylates, polyketals, polyhydroxyvalerates, polyalkylene oxalates, polyalkylene succinates, copolymers thereof, and mixtures thereof.

Biodegradable and biocompatible polymers containing lactic acid units, such as poly-L-lactic acid, poly-D-lactic acid, poly-D, L-lactic acid, poly-L-lactide, poly-D-lactide, and poly-D, L-lactide, are collectively referred to herein as poly(lactic acid) or “PLA.” Those that contain caprolactone units, such as poly(ε-caprolactone), are collectively referred to herein as poly(caprolactone) or “PCL.” Copolymers including lactic acid and glycolic acid units, such as various forms of poly(lactic acid-co-glycolic acid) and poly(lactide-co-glycolide) characterized by the ratio of lactic acid:glycolic acid, are collectively referred to herein as poly(lactic acid-co-glycolic acid) or “PLGA.”

Optionally, the polymer or each polymer in a blend of polymers used to form the core of the coated particle is polystyrene, poly(methyl methacrylate), poly(ethylene glycol), poly(lactic acid), poly[butadiene-co-(maleic anhydride)], poly(butadiene-maleic anhydride-co-L-DOPA), poly(fumaric-co-sebacic acid), poly(glycolic acid), poly(lactic acid-co-glycolic acid), polysebacic acid, polyfumaric acid, or copolymers thereof, or a mixture thereof.

Optionally, the polymer or each polymer in a blend of polymers used to form the core of the coated particle is polystyrene, poly(lactic acid), poly(butadiene-maleic anhydride-co-L-DOPA), poly(fumaric-co-sebacic acid), poly(glycolic acid), or poly(lactic acid-co-glycolic acid).

In some forms, the polymer(s) used to form the core of the coated particle confer(s) the negative charge. Such polymers can be those described above, such as polyesters (such as poly(caprolactone); poly(hydroxy acids), such as poly(lactic acid), poly(glycolic acid), and poly(lactic acid-co-glycolic acids); and/or polyhydroxyalkanoates, such as poly(3-hydroxybutyrate) and poly(4-hydroxybutyrate)) and/or polyanhydrides (such as poly(fumaric-co-sebacic acid), polysebacic acid, and/or polyfumaric acid).

In some forms, the polymer(s) used to form the core of the coated particles contain(s) a moiety that confers negative charge. The negatively charged moiety can be covalently or non-covalently attached to a polymer. For example, the negatively charged moiety is covalently attached to the backbone of the polymer. The moiety can be an acidic group, an anionic group, peptide(s), amino acid(s), lipid(s), salt(s), or combinations thereof. Examples of acidic groups include, but are not limited to, carboxylic acids, protonated sulfates, protonated sulfonates, protonated phosphates, singly- or doubly protonated phosphonates, and singly- or doubly protonated hydroxamate. The corresponding salts of these acidic groups form anionic groups such as carboxylates, sulfates, sulfonates, singly- or doubly deprotonated phosphate, singly- or doubly deprotonated phosphonate, and hydroxamate. Such polymers can be obtained by modifying a polymer of neutral charge to introduce any of the moiety described above.

Optionally, polymers that contain styrene units, such as polystyrene or polystyrene modified by a functional group, such as a negatively charged moiety, are collectively referred to herein as polystyrene or “PS” can form the polymeric core of the coated particle. Coated particles containing PS core can be used for studying the properties of the coated particles.

In some forms, the polymer used to form the core of the coated particle is not or does not contain poly(ethylene glycol). For example, the core is not a polymer to which polyethylene glycol (PEG) is covalently or non-covalently attached. For example, the core is not pegylated, i.e. PEG is not covalently or non-covalently attached to or forming the outer surface of the core.

iii. Molecular Weight

The polymer or each polymer in a blend of polymers that are suitable for forming the core of the coated particles typically has a molecular weight in a range from 1.5 kDa to 300 kDa, from 1.5 kDa to 275 kDa, from 1.5 kDa to 250 kDa, from 1.5 kDa to 100 kDa, from 2 kDa to 80 kDa, from 2 kDa to 50 kDa, from 2 kDa to 30 kDa, from 2 kDa to 20 kDa, or from 2 kDa to 10 kDa

Optionally, the polymeric core of the coated particles contains a blend of a low molecular weight polymer, such as one having a molecular weight in a range from 2 kDa to 20 kDa, from 2 kDa to 15 kDa, or from 2 kDa to 10 kDa, and a high molecular weight polymer, such as one having a molecular weight in a range from 21 kDa to 300 kDa, for example, in the range from greater than 20 kDa to 300 kDa, greater than 20 kDa to 100 kDa, from 25 kDa to 50 kDa, from 30 kDa to 100 kDa, from 100 kDa to 200 kDa, or from 200 kDa to 300 kDa. When the polymeric core of the coated particles contains such a blend, the weight percentage of the low molecular weight polymer in the blend can be in a range from 30% to 90%, such as from 30% to 80%, from 30% to 70%, from 30% to 60%, from 30% to 50%, from 40% to 80%, from 40% to 70%, from 40% to 60%, from 50% to 80%, from 50% to 70%, from 30% to 40%, from 40% to 50%, from 50% to 60%, from 60% to 70%, from 70% to 80%, or from 80% to 90%.

iv. Adhesiveness

Optionally, the polymeric core of the coated particle contains polymers that are bioadhesive. The bioadhesivity of a substance, such as a polymer or coated particle containing such a polymer, refers to the ability of the substance to adhere to a biological surface, such as an epithelial surface, or mucus on an epithelial surface, or both. Typically, adhesion occurs in an aqueous environment. Mucoadhesivity is a more specific form of bioadhesivity that refers to the interaction of a substance and the mucosal tissue. Bioadhesivity can be quantitated in relative terms, such as, but not limited to, a spectrum of bioadhesiveness within a group of substances, such as polymers and/or coated particle containing such a polymer. In some forms where two or more polymers or coated particles containing polymeric cores are being discussed, the terms “bioadhesivity” and “mucoadhesivity” can be defined based on a polymer's or coated particle's relative bioadhesiveness when compared to another, more bioadhesive polymer or coated particle, respectively. Bioadhesivity can be measured as described in Chickering and Mathiowitz, Journal of Controlled Release (1995), 34: 251-261; U.S. Pat. No. 6,197,346 to Mathiowitz, et al.; and U.S. Pat. No. 6,235,313 to Mathiowitz, et al., the contents of which are hereby incorporated by reference.

Suitable bioadhesive polymers for forming the cores of the coated particles can have a bioadhesion force of 100 mN/cm² or higher, such as about 500 mN/cm² (such as 480 mN/cm²), or greater. Methods for measuring bioadhesivity of polymers are known, such as by using a Texture Analyzer containing a pin head coated with the polymer and applying it to an ex-vivo GI segment, such as described in Estrellas, et al., Colloids and Surfaces B: Biointerfaces, 173:454-469 (2019). Examples of such bioadhesive polymers can be those described above, such as polyesters (such as poly(caprolactone); poly(hydroxy acids), such as poly(lactic acid), poly(glycolic acid), and poly(lactic acid-co-glycolic acids); and/or polyhydroxyalkanoates, such as poly(3-hydroxybutyrate) and poly(4-hydroxybutyrate)) and/or polyanhydrides (poly(fumaric-co-sebacic acid), polysebacic acid, polyfumaric acid).

Without being bound by theory, it is believed that using a polymer having an appreciable bioadhesivity to form a polymeric core of a coated particles may contribute to the increased particle systemic uptake by increasing the residence time and adsorption into the mucosal layer.

b. Hydrophobic Drug Core

In some forms, the core of the coated particle is formed of a hydrophobic drug, optionally more than one hydrophobic drug. Generally, the core does not contain excipients, e.g. is substantially free of excipients. For example, the core typically contain less than 1 wt %, less than 0.1 wt %, or less than 0.001 wt % excipients. The hydrophobic drug or each hydrophobic drug of two or more drugs forming the core can be a single molecule of the drug or multiple molecules of the same drug. The hydrophobic drug may be neutral, or negatively or positively charged. The charge of the coated hydrophobic drug may be the same or different from the charge of the hydrophobic drug without a coating. Whether a coating is successfully formed surrounding the drug core may be determined by measuring the diffusion coefficient, hydrodynamic diameter, and/or aggregation of the hydrophobic drug before and after coating.

The term “hydrophobic,” as used herein, refers to the property of lacking affinity for or repelling water. For example, the more hydrophobic a drug, the more that drug tends to not dissolve in, not mix with, or not be wetted by water. Hydrophobicity can be quantified by measuring a drug's partition coefficient between water (or a buffered aqueous solution) and a water-immiscible organic solvent, such as octanol, methylene chloride, or methyl tert-butyl ether. If after equilibration a greater concentration of the drug is attained in the organic solvent than in water, the drug is considered hydrophobic. For example, if the organic solvent is octanol, then a positive log P value indicates that the drug is hydrophobic. “Hydrophobic” may also refer to a drug that when applied to a surface, such as glass, forms a contact angle with water, which is greater than the contact angle of water on a surface of glass without the drug.

The hydrophobic drug(s) coated with a glycoprotein or a combination of a sugar, an oligosaccharide, and/or a polysaccharide, and a suitable protein has/have enhanced diffusivity in mucus (upon administration to a GI tract or other mucosal surfaces, such as vagina in a mammal), compared to the same hydrophobic drug(s) without coating, and thereby achieve(s) increase systemic uptake into the blood stream (systemic).

i. Exemplary Hydrophobic Drugs

Examples of hydrophobic drugs that are suitable for forming the core of the coated particle include, but are not limited to, Biopharmaceutical Classification System (“BCS”) Class IV drugs.

The Biopharmaceutical Classification System (“BCS”), originally developed by G. Amidon, separates pharmaceuticals for oral administration into four classes depending on their aqueous solubility and their permeability through the intestinal cell layer. According to the BCS, drug substances are classified as follows:
Class I—High Permeability, High Solubility
Class II—High Permeability, Low Solubility
Class III—Low Permeability, High Solubility
Class IV—Low Permeability, Low Solubility

The solubility class boundary is based on the highest dose strength of an immediate release (“IR”) formulation and a pH-solubility profile of the test drug in aqueous media with a pH range of 1 to 7.5. Solubility can be measured by the shake-flask or titration method or analysis by a validated stability-indicating assay. A drug substance is considered highly soluble when the highest dose strength is soluble in 250 ml or less of aqueous media over the pH range of 1-7.5. The volume estimate of 250 ml is derived from typical bioequivalence (BE) study protocols that prescribe administration of a drug product to fasting human volunteers with a glass (about 8 ounces) of water.

An IR drug product is considered rapidly dissolving when no less than 85% of the labeled amount of the drug substance dissolves within 30 minutes, using U.S. Pharmacopeia (USP) Apparatus I at 100 rpm (or Apparatus II at 50 rpm) in a volume of 900 ml or less in each of the following media: (1) 0.1 N HCl or Simulated Gastric Fluid USP without enzymes; (2) a pH 4.5 buffer; and (3) a pH 6.8 buffer or Simulated Intestinal Fluid USP without enzymes.

BCS Class IV drugs are lipophilic drugs with poor GI permeability. Examples include acetazolamide, allopurinol, dapsone, doxycycline, paracetamol, nalidixic acid, clorothiazide, tobramycin, cyclosporin, tacrolimus, and paclitaxel. Additional exemplary drugs are drugs for treating COVID-19, such as Molnupiravir and Nirmatrelvir.

ii. Weight Percentage of Hydrophobic Drugs

The weight percentage of the hydrophobic drug or the total weight percentage of the two or more hydrophobic drugs in the coated particle depends on the molecular weights of the hydrophobic drug. Optionally, the weight percentage of the hydrophobic drug or the total weight percentage of the two or more hydrophobic drugs in the coated particle is in a range from 0.01% to 80%, from 0.01% to 60%, from 0.01% to 50%, from 0.01% to 40%, from 0.01% to 25%, from 0.01% to 10%, from 0.01% to 5%, from 0.1% to 50%, from 0.1% to 25%, from 0.1% to 10%, from 0.1% to 5%, from 0.05% to 10%, from 0.05% to 5%, from 0.01% to 2%, from 0.05% to 2%, from 0.1% to 2%, from 0.01% to 1%, from 0.05% to 1%, or from 0.1% to 1%. The term “weight percentage of the hydrophobic drug” refers to the weight of the hydrophobic drug relative to the sum of the weights of the hydrophobic drug, the coating, and optionally any additional active agent(s). The term “total weight percentage of the two or more hydrophobic drugs” refers to the sum of the weights of the hydrophobic drugs relative to the sum of the weights of the hydrophobic drug, the coating, and optionally any additional active agent(s).

The weight percentage of the hydrophobic drug or total weight percentage of the two or more hydrophobic drugs in the coated particle can be varied based on the specific drug(s) being delivered. For example, for hydrophobic biomolecules that form the core of the coated particles, such as hydrophobic lipids, the weight percentages of such hydrophobic biomolecule in the coated particle are from 0 0.01% to 20%, from 0.01% to 5%, from 0.01% to 2.5%, or from 0.01% to 1%. For example, for hydrophobic small molecules that form the core of the coated particles, the weight percentages of such small molecules in the coated particles are from 0.1% to 50%, from 0.1% to 25%, from 0.1% to 10%, from 0.1% to 5%, from 0.1% to 2%, or from 0.1% to 1%.

### 2. Coating

The coating of the coated particles contains a glycoprotein (e.g. mucin, glycoprotein 41, glycoprotein 120, etc.) or a combination of a sugar, an oligosaccharide, and/or a polysaccharide, and a protein that contains an amino acid backbone containing at least 50 repeated units of serine and/or threonine. For example, the amino acid backbone of the protein contains repeated units of serine and/or threonine and the total number of the serine and/or threonine is at least 50. The glycoprotein or the combination of sugar, oligosaccharide, and/or polysaccharide and protein surrounds the core. When the combination of a sugar (e.g. sucrose, glucose, lactose, etc.), oligosaccharide (e.g. oligomer of sucrose, glucose, lactose, etc.), and/or polysaccharide (e.g. polymer of sucrose, glucose, lactose, etc.), and a protein form the coating of the coated particle, the sugar, oligosaccharide, and/or polysaccharide can be covalently attached to or non-covalently associated with the amino acid backbone of the protein.

Glycoproteins contain a variety of molecular interactions, such as disulfide bridging and electrostatic forces, that create a flexible array of alternating hydrophilic/hydrophobic regions. This allows glycoproteins to interact with a core formed by various types of polymers or a hydrophobic drug. Similarly, a combination of sugar, oligosaccharide, and/or polysaccharide, and a protein that contains an amino acid backbone mimicking a glycoprotein, for example an amino acid backbone containing at least 50 repeated units of serine and/or threonine, can also provide a variety of molecular interactions, allowing them to interact with a core formed by various types of polymers or a hydrophobic drug.

The glycoprotein or the combination of sugar, oligosaccharide, and/or polysaccharide and protein surrounds the core. When the combination of a sugar (e.g. sucrose, glucose, lactose, etc.), oligosaccharide (e.g. oligomer of sucrose, glucose, lactose, etc.), and/or polysaccharide (e.g. polymer of sucrose, glucose, lactose, etc.), and a protein form the coating of the coated particle, the sugar, oligosaccharide, and/or polysaccharide can be covalently attached to or non-covalently associated with the amino acid backbone of the protein.

Optionally, the coating includes one or more excipients in a sufficient concentration to improve diffusion of the coated particle. Typically, the total concentration of excipients in the coating is less than 50% (wt), less than 40% (wt), less than 30% (wt), less than 20%, less than 10% (wt), less than 5% (wt), less than 4% (wt), less than 3% (wt), 2% (wt), less than 1% (wt). The term “total concentration of excipients in the coating” refers to the sum of the weight of all excipient(s) in the coating relative to the weight of the coating.

In forms where the core is a polymeric core having a negative or positive zeta potential, the coating of the coated particle is sufficient to reduce the negative or positive charge of the core and bring the zeta potential of the coated particle to near neutral. For example, the coating reduces the negative or positive charge of the polymeric core, bringing the zeta potential of the coated particle to near neutral, such as in a range from −16 mV to 16 mV, from −16 mV to 10 mV, from −16 mV to 5 mV, from −16 mV to 0 mV, from −16 mV to −5 mV, or from −16 mV to −10 mV, in water, at room temperature.

In some forms, the coating of the coated particle is not or does not contain poly(ethylene glycol). For example, the coating is not a glycoprotein to which polyethylene glycol (PEG) is covalently or non-covalently attached. For example, the sugar, oligosaccharide, and/or polysaccharide and protein in a combination thereof do not contain PEG.

### Increased Diffusion Coefficient, Increase Residence Time, Enhanced Transport

In order to penetrate the gastrointestinal tract or the lining of another mucosal surface, orally administered particles or particles that are administered locally to a mucosal surface should diffuse through the mucosal layers at a rate faster than the mucosal clearance rate. Including a glycoprotein coating is one approach used to address this issue since it extends residence time in the tissue.

The importance of residence time can be derived from Equation 1:

$$< {x}^{2} > = 4 \cdot {D}_{m} \cdot t \qquad Equation 1$$

where <x²> is the mean-square displacement, and t is time. The theoretical minimal diffusion coefficient required for a coated particle to diffuse through a mucosal layer can be calculated according to Equation 1. For example, the average residence time in the small intestine of rodents is 2.6-3.3 hours where their jejunum (mucosal layer of ˜120 μm) contains 90% of their small intestine. Therefore, a coated particle with diffusion coefficient (Dₘ) of 2.5-3.0 10⁻⁹ cm²/s should suffice.

The same calculation can be applied to human small intestine where the residence is 3.1-8 hours for fasted and 3-6 hours for fed humans. Human mucosal jejunum thickness ranges from 300 to 400 μm. Thus, for humans, coated particles may have a Dₘ in the range of 3.7-13.9 10⁻⁹ cm²/s or higher. These values are similar to rodents, demonstrating that rodents are a good animal model for evaluating GI uptake. In contrast, pig's mucosal layer of the small intestine is 26-31 μm while its residence time is 2-33 days. Hence, even for the shortest residence time associated with pig's, i.e. a 50 hour residence time, the required Dₘ is 9.4-12.5×10⁻¹² cm²/s. This value is two to three orders of magnitude smaller (even more for 33 days) than the required value for humans and rodents. Thus, pigs are not a good animal model for evaluating GI uptake of coated particles.

Additionally, since the mucous layer is considered a membrane, the permeability coefficient (Pₘ) is suitable for describing penetration through a membrane, as defined in Equation 2:

$${P}_{m} = \frac{{D}_{m} * {K}_{p}}{h} \qquad Equation 2$$

where Kₚ is the partitioning coefficient between the medium and mucosal layer, and h is the mucosal thickness. Therefore, coated particles need to adsorb quickly and in sufficient quantities to the mucosal layer to be effective.

Glycoprotein coatings have a high affinity to the mucosal layer. The affinity of the coated particles to the mucosal layer correlates with Kₚ. Therefore, including a glycoprotein coating can increase both the residence time and extent of mucosal adsorption of the coated particles.

Without being bound by theory, it is believed that GI uptake of the coated particles described herein may follow the chronological order of absorption to the mucosal layer (governed by bioadhesion force), diffusion through the mucosal layer, and GI epithelial cells uptake.

a. Glycoprotein

The coating of the coated particle can be formed from a glycoprotein or more than one glycoprotein. Suitable glycoproteins for forming the coatings of the coated particles are collagen, mucins, glycoprotein 41, glycoprotein 120, transferrin, ceruloplasmin, immunoglobulins, histocompatibility antigens, human chorionic gonadotropin, thyroid-stimulating hormone, enzymes, such as alkaline phosphatase and patatin, proteins involved in cell-cell, virus-cell, bacterium-cell, and/or hormone-cell interactions, plasma proteins of coldwater fish, lectins, selectins, antibodies that interact with carbohydrates, proteins involved in hormone and drug actions, calnexin, calreticulin, notch and its analogs, proteins involved in the regulation of development, glycoproteins on the surface membranes of platelets, and a combination thereof.

In some forms, the coating of the coated particles containing polymeric cores is not or does not contain mucin. In some forms, the coating of the coated particles containing polymeric cores is not or does not contain mucin, at a density that imparts a zeta potential in a range from −16 mV to −7.5 mV to the coated particle. In some forms, the coating of the coated particles containing polymeric cores is not formed by dispersing the polymeric core in a 0.1% (w/v) mucin solution.

i. Exemplary Glycoproteins

Examples of general classes of glycoproteins that are suitable for forming the coating of the coated particle include, but are not limited to, enzymes; proteins involved in cell-cell, virus-cell, bacterium-cell, and/or hormone-cell interactions; plasma proteins of coldwater fish; antibodies that interact with carbohydrates; proteins involved in hormone and drug actions; proteins involved in the regulation of development; and glycoproteins on the surface membranes of platelets.

Specific examples of glycoproteins that are suitable for forming the coating of the coated particle include, but are not limited to, mucin, Glycoprotein-41, Glycoprotein-120, O-linked glycoproteins, N-linked glycoproteins, and nonenzymatic glycosylated proteins. The locant N- is used for the N-glycosyl linkage to asparagine. N-Linked oligosaccharides are divided into two major classes: the N-acetyllactosamine type including N-acetyl-D-glucosamine, D-mannose, D-galactose, L-fucose and sialic acid, and the oligomannose type including N-acetyl-D-glucosamine and a variable number of D-mannose residues. Structures containing both oligomannose- and N-acetyllactosamine-type oligosaccharides are designated as hybrid type. Examples of N-glycoproteins (or N-glycosylproteins) are chicken ovalbumin, pig ribonuclease, human α₁-acid glycoprotein and soybean agglutinin. The locant O- is used for O-glycosyl linkage to serine, threonine, hydroxylysine, or hydroxyproline. Exemplary O-glycoproteins (or O-glycosylproteins) include, but are not limited to sheep submaxillary glycoprotein, collagen, fish antifreeze glycoproteins, and potato lectin. Two types of carbohydrate-peptide linkage in the same protein or peptide chain may be indicated by a combination of the locants. Thus, calf fetuin, procollagen, human erythrocyte membrane glycophorin and human chorionic gonadotropin are N-,O-glycoproteins (or N-,O-glycosylproteins). Additional examples of glycoproteins that are suitable for forming the coating of the coated particles are described in IUPAC-IUB Joint Commission on Biochemical Nomenclature (JCBN), Recommendations 1985, World Wide Web version Prepared by G. P. Moss.

b. Combination of Sugar, Oligosaccharide, and/or Polysaccharide and Protein

Optionally, the coating of the coated particle is formed from a combination of a sugar, an oligosaccharide, and/or a polysaccharide, and a protein that contains an amino acid backbone containing at least 50 repeated units of serine and/or threonine. For example, the protein in a combination with a sugar, an oligosaccharide, and/or a polysaccharide to form the coating of the coated particles contains an amino acid backbone; the amino acid backbone contains repeated units of serine and/or threonine and the total number of serine and/or threonine is at least 50. The sugar in the combination can be bonded covalently or non-covalently to the protein. For example, the sugar in the combination is non-covalently bound to the protein. Exemplary non-covalent bonds between the sugar and the protein include, but are not limited to, electrostatic interactions, affinity interactions, metal coordination, physical adsorption, host-guest interactions, and hydrogen bonding interactions.

The weight percentage of the sugar, oligosaccharide, or polysaccharide, or the total weight percentage of sugar and oligosaccharide, sugar and polysaccharide, oligosaccharide and polysaccharide, or sugar, oligosaccharide, and polysaccharide in the combination of sugar, oligosaccharide, and/or a polysaccharide, and a protein can be in a range from 30% to 90%, from 40% to 80%, or from 50% to 70%. The term “total weight percentage of sugar and oligosaccharide, sugar and polysaccharide, oligosaccharide and polysaccharide,” refers to the sum of the weights of the sugar, oligosaccharide, and/or polysaccharide, relative to the sum of the weights of the sugar, oligosaccharide, and/or polysaccharide, and the protein.

i. Sugar, Oligosaccharide, and Polysaccharide

Examples of suitable sugars, oligosaccharide, and polysaccharide for use in the combination to form the coatings of the coated particles include, but are not limited to, sucrose, glucose, fucose, mannose, galactose, xylose, lactose, maltose, fructose, starch, glycogen, fiber, and a combination thereof, and other oligosaccharides and polysaccharides formed by sucrose, glucose, fucose, mannose, galactose, lactose, maltose, fructose, or xylose, or a combination thereof.

ii. Protein

Generally, proteins that are suitable for use in the combination to form the coatings of the coated particles contain an amino acid backbone containing at least 50 repeated units of serine and/or threonine. For example, the protein in a combination with a sugar, an oligosaccharide, and/or a polysaccharide to form the coating of the coated particles contains an amino acid backbone; the amino acid backbone contains repeated units of serine and/or threonine and the total number of serine and/or threonine is at least 50.

c. Excipients in the Coating

Optionally, the coating includes one or more excipients in a sufficient concentration to improve diffusion of the coated particle. Optionally, the excipients included in the coating also alter one or more additional properties of the coated particles, such as porosity and permeability and/or hydration and disintegration properties. Adjustment of one or more of these additional properties of the coated particles may be used to modify the release rate of the active agent being delivered.

i. Exemplary excipients

Exemplary excipients suitable for use in the coating of the coated particle include, but are not limited to, plasticizers, pigments, colorants, stabilizing agents, glidants, pore formers, and surfactants. Surfactants suitable for use in the coating of the coated particle may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl annonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, Pluronics, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine lauryl sulfobetaine, and lecithin.

Suitable excipients that can alter the porosity and permeability of the coating may include inorganic and organic materials such as sucrose, hydroxypropyl cellulose, sodium chloride, sodium chloride, xylitol, sorbitol, lactose, dextrose, maltodextrins, and dextrates.

Excipients may also be included in the coating to alter its hydration and disintegration properties. Suitable pH dependent enteric excipients may include cellulose acetate phthalate. Excipients may also be added as a “wicking agent” to regulate the hydration of the coating. Suitable excipients may include aedisol, hydroxypropyl cellulose, hydroxypropyl methyl cellulose, and cellulose acetate phthalate.

Poly(adipic acid) may be included in the coating of the coated particles. Poly(adipic acid) prevents coalescence of drug domains within a spray-dried product resulting in increased drug surface area available for dissolution. Additionally, adipic acid monomer generated during polymer degradation increases acidity in the microenvironment of a spray-dried drug particle. By changing the pH, some of the hydrophobic drug and optionally one or more additional active agents included in the coated particles may become more soluble.

ii. Concentration of Excipients in Coating

Typically, the total concentration of excipients in the coating is less than 50% (wt), less than 40% (wt), less than 30% (wt), less than 20%, less than 10% (wt), less than 5% (wt), less than 4% (wt), less than 3% (wt), 2% (wt), less than 1% (wt). For example, the total concentration of excipients in the coating is in a range from about 0.01% (wt) to about 45% (wt), from about 0.05% (wt) to about 45% (wt), from about 0.1% (wt) to about 45% (wt), from about 0.01% (wt) to about 40% (wt), from about 0.05% (wt) to about 40% (wt), from about 0.1% (wt) to about 40% (wt), from about 0.5% (wt) to about 40% (wt), from about 1% (wt) to about 40% (wt), from about 0.01% (wt) to about 30% (wt), from about 0.05% (wt) to about 30% (wt), from about 0.1% (wt) to about 30% (wt), from about 0.5% (wt) to about 30% (wt), from about 1% (wt) to about 30% (wt), from about 0.01% (wt) to about 20% (wt), from about 0.05% (wt) to about 20% (wt), from about 0.1% (wt) to about 20% (wt), from about 0.5% (wt) to about 20% (wt), from about 1% (wt) to about 20% (wt), from about 0.01% (wt) to about 10% (wt), from about 0.05% (wt) to about 10% (wt), from about 0.1% (wt) to about 10% (wt), from about 0.5% (wt) to about 10% (wt), from about 1% (wt) to about 10% (wt), from about 0.01% (wt) to about 5% (wt), from about 0.05% (wt) to about 5% (wt), from about 0.1% (wt) to about 5% (wt), from about 0.01% (wt) to about 1% (wt), from about 0.05% (wt) to about 1% (wt), or from about 0.1% (wt) to about 1% (wt).

### 3. Active Agents

The coated particles may contain an active agent or more than one active agent. When the coated particles contain a polymeric core, the active agents can be encapsulated in the polymeric core and/or embedded in the coating of the coated particles. When the coated particles contain hydrophobic drug(s) as the core, one or more additional active agents, such as a therapeutic agent, diagnostic agent, and/or a prophylactic agent that is different from the hydrophobic drug(s), can be embedded in the coating of the coated particles.

The active agent or each active agent of two or more active agents within the coated particle can be a therapeutic agent, a diagnostic agent, or a prophylactic agent. The active agent(s) is/are delivered to the blood stream (systemic) or to the GI tract or other mucosal surfaces, such as vagina (local) in a mammal via the coated particles. Upon reaching their target locale or systemically, the coated particles can release the active agent(s) in a controlled manner.

Active agents with a wide range of molecular weights can be loaded in the coated particles, for example, between 100 Da and 10,000 kDa. Examples of active agents that can be loaded in the coated particles for delivery include, but are not limited to, small molecules, proteins, polypeptides, peptides, carbohydrates, nucleic acids, glycoproteins, lipids, and antibodies/antigens, and combinations thereof. “Small molecule” generally refers to an organic molecule that is less than about 900 Da. Polypeptide is generally a string of covalently bonded amino acids which are not folded into any specific structure. Typically, small molecules are non-polymeric and/or non-oligomeric. When the active agent loaded in the coated particles is a protein or glycoprotein, the protein or glycoprotein is different from those forming the coating. For example, the coating of the coated particles is formed from a first glycoprotein or a combination of a sugar and a first protein. Such coated particles further contain an active agent, which is optionally a second glycoprotein or a second protein, encapsulated in the polymeric core and/or embedded in the coating of the coated particles. In these forms, if the active agent is or includes a second glycoprotein or second protein, the second glycoprotein or second protein is different from the first glycoprotein or first protein forming the coating of the coated particles.

The weight percentage of the active agent or the total weight percentage of the two or more active agents in the coated particle, when present, depends on the hydrophilicity/hydrophobicity and the molecular weights of the active agents. Optionally, the weight percentage of the active agent or the total weight percentage of the two or more active agents in the coated particle is in a range from 0.01% to 80%, from 0.01% to 60%, from 0.01% to 50%, from 0.01% to 40%, from 0.01% to 25%, from 0.01% to 10%, from 0.01% to 5%, from 0.1% to 50%, from 0.1% to 25%, from 0.1% to 10%, from 0.1% to 5%, from 0.05% to 10%, from 0.05% to 5%, from 0.01% to 2%, from 0.05% to 2%, from 0.1% to 2%, from 0.01% to 1%, from 0.05% to 1%, or from 0.1% to 1%. The term “weight percentage of the active agent” refers to the weight of the active agent relative to the sum of the weights of the active agent, the coating, and the core. The term “total weight percentage of the two or more active agents” refers to the sum of the weights of the active agents relative to the sum of the weights of the active agents, the coating, and the core.

The weight percentage of the active agent or total weight percentage of the two or more active agents can be varied based on the specific agent(s) being delivered. For example, for large biomolecules, such as proteins and nucleic acids, typical weight percentages of the large biomolecule in the coated particle are from 0 0.01% to 20%, from 0.01% to 5%, from 0.01% to 2.5%, or from 0.01% to 1%.

a. Exemplary Active Agents

Examples of active agents and their alternative forms such as alternative salt forms, free acid forms, free base forms, and hydrates that can be loaded in the coated particles for delivery include, but are not limited to, the categories described below. Any of these active agents or a combination thereof can be loaded in the coated particles for delivery.

i. Anticancer Agents

Examples of anticancer agents that can be loaded in the coated particles for delivery include, but are not limited to, 5-fluorouracil; gemcitabine; gemcitabine hydrochloride; cytarabine; decitabine; leucovorin; acivicin, aclarubicin, acodazole hydrochloride, acronine, adozelesin, aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; dacarbazine; dactinomycin; daunorubicin hydrochloride; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; flurocitabine; fosquidone; fostriecin sodium; hydroxyurea; idarubicin hydrochloride; ifosfamide; ilmofosine; interleukin II (including recombinant interleukin II, orrIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimus tine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol,9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; HMG-CoA reductase inhibitor (such as but not limited to, Lovastatin, Pravastatin, Fluvastatin, Statin, Simvastatin, and Atorvastatin); loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidasc; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosinc; superactive vasoactive intestinal peptide antagonist; suradista; suranin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stern cell factor; translation inhibitors; tretinoin; triacetyluridine; triciribinc; trimetrexate; triptorelin; tropisetron; turosteridc; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; Vitaxin®; vorozole; zanotcrone; zeniplatin; zilascorb; and zinostatin stimalamer.

ii. Analgesics/Antipyretics

Examples of analgesics/antipyretics that can be loaded in the coated particles for delivery include, but are not limited to, aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate); and antiasthamatics (e.g., ketotifen and traxanox).

iii. Antibiotics

Examples of antibiotics that can be loaded in the coated particles for delivery include, but are not limited to, neomycin, streptomycin, chloramphenicol, cephalosporin, ampicillin, penicillin, tetracycline, and ciprofloxacin).

iv. Antidepressants

Examples of antidepressants that can be loaded in the coated particles for delivery include, but are not limited to, nefopam, oxypertine, doxepin, amoxapine, trazodone, amitriptyline, maprotiline, phenelzine, desipramine, nortriptyline, tranylcypromine, fluoxetine, doxepin, imipramine, imipramine pamoate, isocarboxazid, trimipramine, and protriptyline).

v. Antidiabetics

Examples of antidiabetics that can be loaded in the coated particles for delivery include, but are not limited to, biguanides and sulfonylurea derivatives.

vi. Antifungal Agents

Examples of antifungal agents that can be loaded in the coated particles for delivery include, but are not limited to, griseofulvin, ketoconazole, itraconizole, amphotericin B, nystatin, and candicidin.

vii. Antihypertensive Agents

Examples of antihypertensive agents that can be loaded in the coated particles for delivery include, but are not limited to, propranolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine.

viii. Anti-Inflammatories

Examples of anti-inflammatories that can be loaded in the coated particles for delivery include, but are not limited to, non-steroidal anti-inflammatories such as indomethacin, ketoprofen, flurbiprofen, naproxen, ibuprofen, ramifenazone, piroxicam, and steroidal anti-inflammatories such as cortisone, dexamethasone, deflazacort, celecoxib, rofecoxib, hydrocortisone, prednisolone, and prednisone).

ix. Antianxiety Agents

Examples of antianxiety agents that can be loaded in the coated particles for delivery include, but are not limited to, lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene.

x. Immunosuppressive Agents

Examples of immunosuppressive agents that can be loaded in the coated particles for delivery include, but are not limited to, cyclosporine, azathioprine, mizoribine, and FK506 (tacrolimus).

xi. Antimigraine Agents

Examples of antimigraine agents that can be loaded in the coated particles for delivery include, but are not limited to, ergotamine, propranolol, isometheptene mucate, and dichloralphenazone.

xii. Sedatives/Hypnotics

Examples of sedatives/hypnotics that can be loaded in the coated particles for delivery include, but are not limited to, barbiturates such as pentobarbital, pentobarbital, and secobarbital; and benzodiazapines such as flurazepam hydrochloride, triazolam, and midazolam.

xiii. Antianginal Agents

Examples of antianginal agents that can be loaded in the coated particles for delivery include, but are not limited to, beta-adrenergic blockers; calcium channel blockers such as nifedipine, and diltiazem; and nitrates such as nitroglycerin, isosorbide dinitrate, pentaerythritol tetranitrate, and erythrityl tetranitrate.

xiv. Antipsychotic Agents

Examples of antipsychotic agents that can be loaded in the coated particles for delivery include, but are not limited to, haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, and prochlorperazine).

xv. Antimanic Agents

Examples of antimanic agents that can be loaded in the coated particles for delivery include, but are not limited to, lithium carbonate.

xvi. Antiarrhythmics

Examples of antiarrhythmics that can be loaded in the coated particles for delivery include, but are not limited to, bretylium tosylate, esmolol, verapamil, amiodarone, encainide, digoxin, digitoxin, mexiletine, disopyramide phosphate, procainamide, quinidine sulfate, quinidine gluconate, quinidine polygalacturonate, flecainide acetate, tocainide, and lidocaine.

xvii. Antiarthritic Agents

Examples of antiarthritic agents that can be loaded in the coated particles for delivery include, but are not limited to, phenylbutazone, sulindac, penicillamine, salsalate, piroxicam, azathioprine, indomethacin, meclofenamate, gold sodium thiomalate, ketoprofen, auranofin, aurothioglucose, and tolmetin sodium

xviii. Antigout Agents

Examples of antigout agents that can be loaded in the coated particles for delivery include, but are not limited to, colchicine, and allopurinol.

xix. Anticoagulants

Examples of anticoagulants that can be loaded in the coated particles for delivery include, but are not limited to, heparin, heparin sodium, and warfarin sodium.

xx. Thrombolytic Agents

Examples of thrombolytic agents that can be loaded in the coated particles for delivery include, but are not limited to, urokinase, streptokinase, and alteplase.

xxi. Antifibrinolytic Agents

Examples of antifibrinolytic agents that can be loaded in the coated particles for delivery include, but are not limited to, aminocaproic acid.

xxii. Hemorhcologic Agents

Examples of hemorheologic agents that can be loaded in the coated particles for delivery include, but are not limited to, pentoxifylline.

xxiii. Antiplatelet Agents

Examples of antiplatelet agents that can be loaded in the coated particles for delivery include, but are not limited to, aspirin.

xxiv. Anticonvulsants

Examples of anticonvulsants that can be loaded in the coated particles for delivery include, but are not limited to, valproic acid, divalproex sodium, phenytoin, phenytoin sodium, clonazepam, primidone, phenobarbitol, carbamazepine, amobarbital sodium, methsuximide, metharbital, mephobarbital, mephenytoin, phensuximide, paramethadione, ethotoin, phenacemide, secobarbitol sodium, clorazepate dipotassium, and trimethadione.

xxv. Antiparkinson Agents

Examples of antiparkinson agents that can be loaded in the coated particles for delivery include, but are not limited to, ethosuximide.

xxvi. Antihistamines/Antipruritics

Examples of antihistamines/antipruritics that can be loaded in the coated particles for delivery include, but are not limited to, hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, and methdilazine.

xxvii. Agents for Calcium Regulation

Examples of agents useful for calcium regulation that can be loaded in the coated particles for delivery include, but are not limited to, calcitonin, and parathyroid hormone.

xxviii. Antibacterial Agents

Examples of antibacterial agents that can be loaded in the coated particles for delivery include, but are not limited to, amikacin sulfate, aztreonam, chloramphenicol, chloramphenicol palmitate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate.

xxix. Antiviral Agents

Examples of antiviral agents that can be loaded in the coated particles for delivery include, but are not limited to, interferon alpha, beta or gamma, zidovudine, amantadine hydrochloride, ribavirin, and acyclovir.

xxx. Antimicrobials

Examples of antimicrobials that can be loaded in the coated particles for delivery include, but are not limited to, cephalosporins such as cefazolin sodium, cephradine, cefaclor, cephapirin sodium, ceftizoxime sodium, cefoperazone sodium, cefotetan disodium, cefuroxime e azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium; penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, and nafcillin sodium; erythromycins such as erythromycin ethylsuccinate, erythromycin, erythromycin estolate, erythromycin lactobionate, erythromycin stearate, and erythromycin ethylsuccinate; and tetracyclines such as tetracycline hydrochloride, doxycycline hyclate, and minocycline hydrochloride, azithromycin, and clarithromycin.

xxxi. Anti-Infectives

Examples of anti-infectives that can be loaded in the coated particles for delivery include, but are not limited to, GM-CSF.

xxxii. Bronchodilators

Examples of bronchodilators that can be loaded in the coated particles for delivery include, but are not limited to, sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; inhalant corticosteroids such as beclomethasone dipropionate (BDP), and beclomethasone dipropionate monohydrate; salbutamol; ipratropium bromide; budesonide; ketotifen; salmeterol; xinafoate; terbutaline sulfate; triamcinolone; theophylline; nedocromil sodium; metaproterenol sulfate; albuterol; flunisolide; and fluticasone proprionate.

xxxiii. Steroidal Compounds, Hormones and Hormone Analogues

Examples of steroidal compounds, hormones and hormone analogues that can be loaded in the coated particles for delivery include, but are not limited to, incretins and incretin mimetics such as glucagon-like peptide-1 (“GLP-1”), GLP-1 analogs, exenatide, liraglutide, lixisenatide, albiglutide, dulaglutide, semaglutide, taspoglutide, androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium.

xxxiv. Hypoglycemic Agents

Examples of hypoglycemic agents that can be loaded in the coated particles for delivery include, but are not limited to, human insulin, purified beef insulin, purified pork insulin, recombinantly produced insulin, insulin analogs, glyburide, chlorpropamide, glipizide, tolbutamide, and tolazamide);

xxxv. Hypolipidemic Agents

Examples of hypolipidemic agents that can be loaded in the coated particles for delivery include, but are not limited to, clofibrate, dextrothyroxine sodium, probucol, pravastitin, atorvastatin, lovastatin, and niacin.

xxxvi. Peptides, Proteins, and Nucleic Acids

Examples of peptides, proteins, and nucleic acids that can be loaded in the coated particles for delivery include, but are not limited to, DNase, alginase, superoxide dismutase, lipase, sense or anti-sense nucleic acids encoding any therapeutically useful protein, including any of the proteins described herein, and siRNA.

When the active agent being delivered is a large protein, the protein is at least 100 kDa, at least 110 kDa, at least 120 kDa, at least 130 kDa, at least 140 kDa, at least 150 kDa, etc., up to about 10,000 kDa. However, large molecular weight proteins are generally in the range of about 100 kDa or 150 kDa or 200 kDa up to about 1,500 kDa, or about 1,000 kDa.

In some forms, the protein being delivered in the coated particle is an antibody. The term “antibody” is intended to denote an immunoglobulin molecule that possesses a variable region antigen recognition site. The term “variable region” is intended to distinguish such domain of the immunoglobulin from domains that are broadly shared by antibodies (such as an antibody Fc domain). The variable region includes a hypervariable region whose residues are responsible for antigen binding. The hypervariable region includes amino acid residues from a Complementarity Determining Region or CDR (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a hypervariable loop (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). Framework Region or FR residues are those variable domain residues other than the hypervariable region residues as herein defined.

The term “antibody” includes monoclonal antibodies, multispecific antibodies, human antibodies, humanized antibodies, synthetic antibodies, chimeric antibodies, camelized antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies). In particular, such antibodies include immunoglobulin molecules of any type (e.g., IgG, IgE, IgM, IgD, IgA, and IgY), class (e.g., IgG₁, IgG₂, IgG₃, IgG₄, IgA₁, and IgA₂) or subclass. Thus, the term “antibody” includes both intact molecules as well as fragments thereof that include the antigen-binding site and are capable of binding to the desired epitope. These include Fab and F(ab′)₂ fragments which lack the Fc fragment of an intact antibody, and therefore clear more rapidly from the circulation, and may have less non-specific tissue binding than an intact antibody (Wahl et al., J. Nuc. Med. 24:316-325 (1983)). Also included are Fv fragments (Hochman, J. et al., Biochemistry, 12:1130-1135(1973); Sharon, J. et al., Biochemistry, 15:1591-1594 (1976)).

In some forms, the antibody being delivered in the coated particle is a therapeutic antibody. Suitable therapeutic antibodies include, but are not limited to, those discussed in Reichert, Mabs, 3(1): 76-99 (2011), for example, AIN-457, bapineuzumab, brentuximab vedotin, briakinumab, dalotuzumab, epratuzumab, farletuzumab, girentuximab (WX-G250), naptumomab estafenatox, necitumumab, obinutuzumab, otelixizumab, pagibaximab, pertuzumab, ramucirumab, REGN88, reslizumab, solanezumab, T1h, teplizumab, trastuzumab emtansine, tremelimumab, vedolizumab (ENTYVIO®), zalutumumab and zanolimumab.

Other therapeutic antibodies approved for use in clinical trials or in development for clinical use that can be included in the coated particle for delivery include, but are not limited to, rituximab (Rituxan®, IDEC/Genentech/Roche) (see for example U.S. Pat. No. 5,736,137), a chimeric anti-CD20 antibody approved to treat Non-Hodgkin's lymphoma; HuMax-CD20, an anti-CD20 currently being developed by Genmab, an anti-CD20 antibody described in U.S. Pat. No. 5,500,362, AME-133 (Applied Molecular Evolution), hA20 (Immunomedics, Inc.), HumaLYM (Intracel), and PR070769 (PCT/US2003/040426, entitled “Immunoglobulin Variants and Uses Thereof”), trastuzumab (Herceptin®, Genentech) (see for example U.S. Pat. No. 5,677,171), a humanized anti-Her2/neu antibody approved to treat breast cancer; pertuzumab (rhuMab-2C4, Omnitarge), currently being developed by Genentech; an anti-Her2 antibody described in U.S. Pat. No. 4,753,894; cetuximab (Erbitux®, Imclone) (U.S. Pat. No. 4,943,533; PCT WO 96/40210), a chimeric anti-EGFR antibody in clinical trials for a variety of cancers; ABX-EGF (U.S. Pat. No. 6,235,883), currently being developed by Abgenix-Immunex-Amgen; HuMax-EGFr (U.S. Ser. No. 10/172,317), currently being developed by Genmab; 425, EMD55900, EMD62000, and EMD72000 (Merck KGaA) (U.S. Pat. No. 5,558,864; Murthy et al. 1987, Arch Biochem Biophys. 252(2):549-60; Rodeck et al., 1987, J Cell Biochem. 35(4):315-20; Kettleborough et al., 1991, Protein Eng. 4(7):773-83); 1CR62 (Institute of Cancer Research) (PCT WO 95/20045; Modjtahedi et al., 1993, J. Cell Biophys. 1993, 22(1-3):129-46; Modjtahedi et al., 1993, Br J Cancer. 1993, 67(2):247-53; Modjtahedi et al, 1996, Br J Cancer, 73(2):228-35; Modjtahedi et al, 2003, Int J Cancer, 105(2):273-80); TheraCIM hR3 (YM Biosciences, Canada and Centro de Immunologia Molecular, Cuba (U.S. Pat. Nos. 5,891,996; 6,506,883; Mateo et al, 1997, Immunotechnology, 3(1):71-81); mAb-806 (Ludwig Institue for Cancer Research, Memorial Sloan-Kettering) (Jungbluth et al. 2003, Proc Natl Acad Sci USA. 100(2):639-44); KSB-102 (KS Biomedix); MRI-1 (IVAX, National Cancer Institute) (PCT WO 0162931A2); and SC100 (Scancell) (PCT WO 01/88138); alemtuzumab (Campath®, Millenium), a humanized mAb currently approved for treatment of B-cell chronic lymphocytic leukemia; muromonab-CD3 (Orthoclone OKT3®), an anti-CD3 antibody developed by Ortho Biotech/Johnson & Johnson, ibritumomab tiuxetan (Zevalin®), an anti-CD20 antibody developed by IDEC/Schering AG, gemtuzumab ozogamicin (Mylotarg®), an anti-CD33 (p67 protein) antibody developed by Celltech/Wyeth, alefacept (Amevive®), anti-LFA-3 Fc fusion developed by Biogen), abciximab (ReoPro®), developed by Centocor/Lilly, basiliximab (Simulect®), developed by Novartis, palivizumab (Synagis®), developed by Medimmune, infliximab (Remicade®), an anti-TNFalpha antibody developed by Centocor, adalimumab (Humira®), an anti-TNFalpha antibody developed by Abbott, Humicade®, an anti-TNFalpha antibody developed by Celltech, golimumab (CNTO-148), a fully human TNF antibody developed by Centocor, etanercept (Enbrel®), an p75 TNF receptor Fc fusion developed by Immunex/Amgen, lenercept, an p55TNF receptor Fc fusion previously developed by Roche, ABX-CBL, an anti-CD 147 antibody being developed by Abgenix, ABX-IL8, an anti-IL8 antibody being developed by Abgenix, ABX-MAI, an anti-MUC18 antibody being developed by Abgenix, Pemtumomab (R1549,90Y-muHMFG1), an anti-MUC1 in development by Antisoma, Therex (R1550), an anti-MUC1 antibody being developed by Antisoma, AngioMab (AS1405), being developed by Antisoma, HuBC-1, being developed by Antisoma, Thioplatin (AS1407) being developed by Antisoma, Antegrene (natalizumab), an anti-alpha-4-beta-1 (VLA-4) and alpha-4-beta-7 antibody being developed by Biogen, VLA-1 mAb, an anti-VLA-1 integrin antibody being developed by Biogen, LTBR mAb, an anti-lymphotoxin beta receptor (LTBR) antibody being developed by Biogen, CAT-152, an anti-TGF-.beta.2 antibody being developed by Cambridge Antibody Technology, ABT 874 (J695), an anti-IL-12 p40 antibody being developed by Abbott, CAT-192, an anti-TGF.beta.1 antibody being developed by Cambridge Antibody Technology and Genzyme, CAT-213, an anti-Eotaxinl antibody being developed by Cambridge Antibody Technology, LyntphoStat-B® an anti-Blys antibody being developed by Cambridge Antibody Technology and Human Genome Sciences Inc., TRAIL-R1mAb, an anti-TRAIL-R1 antibody being developed by Cambridge Antibody Technology and Human Genome Sciences, Inc. Avastin® bevacizumab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech. Xolair® (Omalizurnab), an anti-IgE antibody being developed by Genentech, Raptiva® (Efalizurnab), an anti-CD11a antibody being developed by Genentech and Xoma, MLN-02 Antibody (formerly LDP-02), being developed by Genentech and Millenium Pharmaceuticals, HuMax CD4, an anti-CD4 antibody being developed by Genmab, HuMax-I_15, an anti-IL15 antibody being developed by Genmab and Amgen, HuMax-Inflam, being developed by Genmab and Medarex, HuMax-Cancer, an anti-Heparanase I antibody being developed by Genmab and Medarex and Oxford GcoSciences, HuMax-Lymphoma, being developed by Genmab and Amgen, HuMax-TAC, being developed by Genmab, IDEC-131, and anti-CD40L antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDFC Pharmaceuticals, IDEC-152, an anti-CD23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-Cide® (labetuzumab), an anti-carcinoembryonic antigen (CEA) antibody being developed by Immunomedics, LymphoCide® (Epratuzumab), an anti-CD22 antibody being developed by Immunomedics, AFP-Cide, being developed by Immunomedics, MyelomaCide, being developed by Immunomedics, LkoCide, being developed by Immunomedics, ProstaCide, being developed by Immunomedics, MDX-010, an anti-CTLA4 antibody being developed by Medarex, MDX-060, an anti-CD30 antibody being developed by Medarex, MDX-070 being developed by Medarex, MDX-018 being developed by Medarex, Osidem® (IDM-I), and anti-Her2 antibody being developed by Medarex and Immuno-Designed Molecules, HuMaxe-CD4, an anti-CD4 antibody being developed by Medarex and Genmab, HuMax-IL15, an anti-IL15 antibody being developed by Medarex and Genmab, CNTO 148, an anti-TNFα antibody being developed by Medarex and Centocor/J&J. CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, Nuvion® (visilizumab), an anti-CD3 antibody being developed by Protein Design Labs, HuZAFO, an anti-gamma interferon antibody being developed by Protein Design Labs, Anti-501 Integrin, being developed by Protein Design Labs, anti-IL-12, being developed by Protein Design Labs, ING-1, an anti-Ep-CAM antibody being developed by Xoma, Xolair® (Omalizumab) a humanized anti-IgE antibody developed by Genentech and Novartis, and MLNO1, an anti-Beta2 integrin antibody being developed by Xoma. In another embodiment, the therapeutics include KRN330 (Kirin); huA 33 antibody (A33, Ludwig Institute for Cancer Research); CNTO 95 (alpha V integrins, Centocor); MEDI-522 (alpha V133 integrin, Medimmune); volociximab (αVβ1 integrin, Biogen/PDL); Human mAb 216 (B cell glycosolated epitope, NCI); BiTE MT103 (bispecific CD19x CD3, Medimmune); 4G7x H22 (Bispecific BcellxFcgammaR1, Meclarex/Merck KGa); rM28 (Bispecific CD28×MAPG, EP1444268); MDX447 (EMD 82633) (Bispecific CD64×EGFR, Medarex); Catumaxomab (removah) (Bispecific EpCAM x anti-CD3, Trion/Fres); Ertumaxomab (bispecific HER2/CD3, Fresenius Biotech); oregovomab (OvaRex) (CA-125, ViRexx); Rencarex® (WX G250) (carbonic anhydrase IX, Wilex); CNTO 888 (CCL2, Centocor); TRC105 (CD105 (endoglin), Tracon); BMS-663513 (CD137 agonist, Brystol Myers Squibb); MDX-1342 (CD19, Medarex); Siplizumab (MEDI-507) (CD2, Medimmune); Ofatumumab (Humax-CD20) (CD20, Genmab); Rituximab (Rituxan) (CD20, Genentech); THIOMAB (Genentech); veltuzumab (hA20) (CD20, Immunomedics); Epratuzumab (CD22, Amgen); lumiliximab (IDEC 152) (CD23, Biogen); muromonab-CD3 (CD3, Ortho); HuM291 (CD3 fc receptor, PDL Biopharma); HeFi-1, CD30, NCI); MDX-060 (CD30, Medarex); MDX-1401 (CD30, Medarex); SGN-30 (CD30, Seattle Genentics); SGN-33 (Lintuzumab) (CD33, Seattle Genentics); Zanolimumab (HuMax-CD4) (CD4, Genmab); HCD 122 (CD40, Novartis); SGN-40 (CD40, Seattle Genentics); Campathlh (Alemtuzumab) (CD52, Genzyme); MDX-1411 (CD70, Medarex); hLL1 (EPB-I) (CD74.38, Immunomedics); Galiximab (IDEC-144) (CD80, Biogen); MT293 (TRC093/D93) (cleaved collagen, Tracon); HuLuc63 (CS1, PDL Pharma); ipilimumab (MDX-010) (CTLA4, Brystol Myers Squibb); Tremelimumab (Ticilimumab, CP-675,2) (CTLA4, Pfizer); 1-IGS-ETR1 (Mapatumumab) (DR4TRAIL-R1 agonist, Human Genome Science/Glaxo Smith Kline); AMG-655 (DR5, Amgen); Apomab (DR5, Genentech); CS-1008 (DR5, Daiichi Sankyo); HGS-ETR2 (lexatumumab) (DR5TRAIL-R2 agonist, HGS); Cetuximab (Erbitux) (EGFR, Imelone); IMC-11F8, (EGFR, Imelone); Nimotuzumab (EGFR, YM Bio); Panitumumab (Vectabix) (EGFR, Amgen); Zalutumumab (HuMaxEGFr) (EGFR, Genmab); CDX-110 (EGFRvIII, AVANT Immunotherapeutics); adecatumumab (MT201) (Epcam, Merck); edrecolomab (Panorex, 17-1A) (Epeam Glaxo/Centocor); MORAb-003 (folate receptor a, Morphotech); KW-2871 (ganglioside GD3, Kyowa); MORAb-009 (GP-9, Morphotech); CDX-1307 (MDX-1307) (hCGb, Celldex); Trastuzumab (Herceptin) (HER2, Celldex); Pertuzumab (rhuMAb 2C4) (HER2 (DI), Genentech); apolizumab (HLA-DR beta chain, PDL Pharma); AMG-479 (IGF-1R, Amgen); anti-IGF-1R R1507 (IGF1-R, Roche); CP 751871 (IGF 1-R, Pfizer); IMC-A12 (IGF1-R, Imclone); B1111022 Biogen); Mik-beta-1 (IL-2Rb (CD122), Hoffman LaRoche); CNTO 328 (IL6, Centocor); Anti-KIR (1-7F9) (Killer cell Ig-like Receptor (KIR), Novo); Hu3S193 (Lewis (y), Wyeth, Ludwig Institute of Cancer Research); hCBE-11 (LTOR, Biogen); HuHMFG1 (MUC1, Antisoma/NCI); RAV 12 (N-linked carbohydrate epitope, Raven); CAL (parathyroid hormone-related protein (PTH-rP), University of California); CT-011 (PD1, CtireTech); MDX-1106 (ono-4538) (PDL Nileclarox/Ono); MAb CT-011(PD1, Curetech); IMC-3G3 (PDGFRa, Imclone); bavituximab (phosphatidylserine, Peregrine); huJ591 (PSMA, Cornell Research Foundation); muJ591 (PSMA, Cornell Research Foundation); GC1008 (TGFb (pan) inhibitor (IgG4), Genzyme); Infliximab (Remicade) (TNFα, Centocor); A27.15 (transferrin receptor, Salk Institute, INSERN WO 2005/111082); E2.3 (transferrin receptor, Salk Institute); Bevacizumab (Avastin) (VEGF, Genentech); HuMV833 (VEGF, Tsukuba Research Lab-WO/2000/034337, University of Texas); IMC-18F1 (VEGFR1, Imclone); IMC-1121 (VEGFR2, Imelone).

xxxvii. Agents for Erythropoiesis Stimulation

Examples of agents useful for erythropoiesis that can be loaded in the coated particles for delivery include, but are not limited to, erythropoietin.

xxxviii. Antiulcer/Anti-Reflux Agents

Examples of antiulcer/anti-reflux agents that can be loaded in the coated particles for delivery include, but are not limited to, famotidine, cimetidine, and ranitidine hydrochloride.

xxxvix. Antinauseants/Antiemetics

Examples of antinauseants/antiemetics that can be loaded in the coated particles for delivery include, but are not limited to, meclizine hydrochloride, nabilone, prochlorperazine, dimenhydrinate, promethazine hydrochloride, thiethylperazine, and scopolamine.

xxxx. Vitamins

Examples of vitamins that can be loaded in the coated particles for delivery include, but are not limited to, vitamins A, D, E, K, and the like; as well as other drugs such as mitotane, halonitrosoureas, anthrocyclines, and ellipticine.

A description of these and other classes of useful agents and a listing of species within each class can be found in Martindale, The Extra Pharmacopoeia, 30th Ed. (The Pharmaceutical Press, London 1993), the disclosure of which is incorporated herein by reference in its entirety.

### 4. Properties

The performance of the coated particles for delivery of hydrophobic drugs (being the core) and/or active agent(s) can be evaluated by the properties of these particles, such as diffusion coefficient, aggregation, and systemic uptake.

a. Diffusion Coefficient

The coated particles can have a diffusion coefficient in mucus or a mucin solution (e.g. 3%-7% w/v mucin, such as 5% w/v mucin) that is greater than the diffusion coefficient of control particles in the same mucus or mucin solution, under the same measurement conditions. Optionally, the coated particles also show a reduced particle aggregation in mucus or a mucin solution (e.g. 3%-7% w/v mucin, such as 5% w/v mucin), as shown by a reduced number of clusters detected, compared to the number of clusters detected for control particles in the same mucus or mucin solution, under the same measurement conditions.

In some forms, the coated particles have a diffusion coefficient in mucus or a mucin solution (e.g. 3%-7% w/v mucin, such as 5% w/v mucin) that is greater than the diffusion coefficient of control particles containing the core only in water, under the same measurement conditions.

In some forms, the control particles containing the core only have a diffusion coefficient in mucus or a mucin solution (e.g. 3%-7% w/v mucin, such as 5% w/v mucin) that is greater than the diffusion coefficient of the control particles in water, under the same measurement conditions.

In some forms, the diffusion coefficient of the coated particles in mucus or a mucin solution (e.g., 3%-7% w/v mucin, such as 5% w/v mucin) has a first enhancement factor, compared to the diffusion coefficient of control particles containing the core only in water; and the diffusion coefficient of the same control particles in mucus or a mucin solution (e.g. 3%-7% w/v mucin, such as 5% w/v mucin) has a second enhancement factor, compared to the diffusion coefficient of the same control particles in water.

In these forms, the first enhancement factor is greater than the second enhancement factor.

Based on the Stokes-Einstein equation, particles were expected to diffuse faster in water than in mucin solutions:

$$D = \frac{{k}_{B} T}{6 π η r}$$

D is diffusion coefficient, k_{B} is Boltzmann's constant, T is temperature (Kelvin), η is dynamic viscosity, and r is the radius of the spherical particle.

The addition of mucin to an aqueous solution is expected to result in both increased viscosity and increased particle size. The increased viscosity (η) and particle size would result in a decreased diffusion coefficient of particles as the Stokes-Einstein equation is inversely proportional to these parameters. Since mucin has an increased viscosity compared to water and can result in increased particle size due to coating, both of these increases would suggest slower diffusion in mucin compared to water. However, the opposite was observed. Thus, the enhanced diffusion coefficient of coated/control particles in mucus or a mucin solution compared to the coated/control particles in water, under the same measurement conditions, does not follow the prediction using Stokes-Einstein equation.

Methods for measuring the diffusivity of the coated particles or control particles in mucus or water are known, for example, by taking time-lapse images of the coated/control particles in a mucus sample or water using a laser microscope and analyzing these images using multiple ParticleTracker and/or particle analyzer add-on(s) in ImageJ. Dynamic light scattering (DLS) can be used to measure diffusivity in water and low concentrations of mucin, such as 0.1% w/v mucin solutions or less concentrated mucin solutions. An exemplary method for measuring and determining the diffusion coefficient of the coated particles using Brownian motion includes: (i) adding the coated nanoparticles or control particles into a mucin solution, typically a 3%-7% w/v mucin solution, which mimics the mucous membrane (known to contain 3-7% w/v of mucin); (ii) capturing time-lapse confocal images at ≥10 different locations; and (iii) performing analysis of the images captured in step (ii) using multiple ParticleTracker add-on for imageJ to determine the diffusion coefficient of the coated particles or control particles. For individual particles, selected regions of interest in each of the images are analyzed to obtain an average diffusion coefficient. A region of interest analysis does not include clusters when calculating the average diffusion coefficient.

Alternatively, full images can be analyzed and will include clusters into the calculation of average diffusion coefficient. If needed, full images are first contrast enhanced, smoothed, sharpened, and/or made binary for cluster analysis. When full images of particles are analyzed, a distribution curve of particles' diffusion coefficients is obtained. A geometric mean value of the distribution curve represents the diffusion coefficient of the entire population of particles being analyzed.

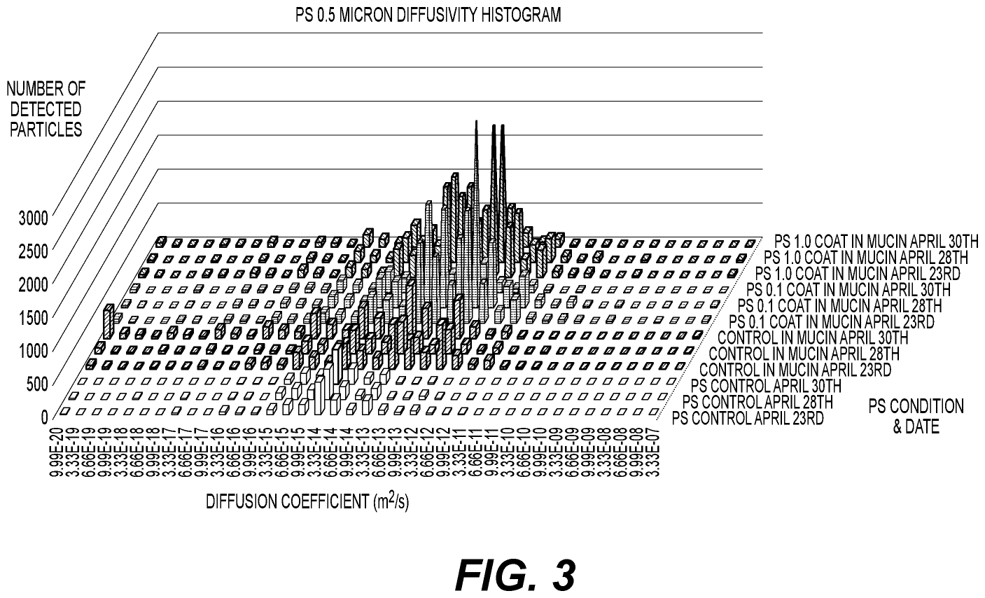

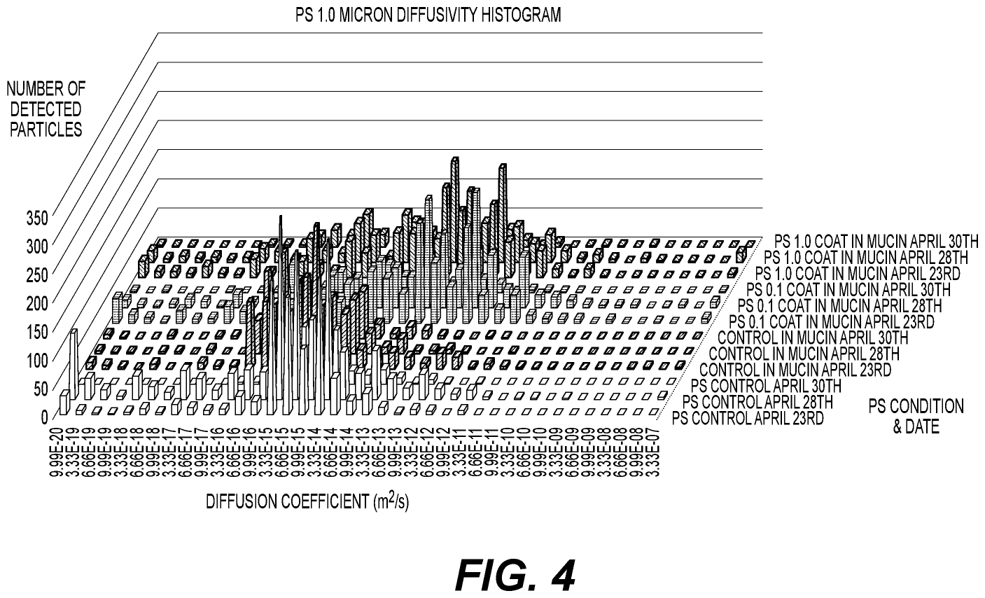

For example, the coated particles have a diffusion coefficient in mucus or a mucin solution (e.g. 3%-7% w/v mucin, such as 5% w/v mucin) that is greater than the diffusion coefficient of control particles in the same mucus or mucin solution, under the same measurement conditions, as shown by the peak or geometric mean value of the distribution curves of the particles' diffusion coefficients (see, e.g., FIGS. 3 and 4, “Control in Mucin” vs. “PS 0.1/1.0 Coat in Mucin”).

For example, the coated particles have a diffusion coefficient in mucus or a mucin solution (e.g. 3%-7% w/v mucin, such as 5% w/v mucin) that is greater than the diffusion coefficient of the control particles in water, under the same measurement conditions, as shown by the peak or geometric mean value of the distribution curves of the coated or control particles' diffusion coefficients (see, e.g., FIGS. 3 and 4, “PS Control” vs. “PS 0.1/1.0 Coat in Mucin”).

For example, the control particles have a diffusion coefficient in mucus or a mucin solution (e.g. 3%-7% w/v mucin, such as 5% w/v mucin) that is greater than the diffusion coefficient of the control particles in water, under the same measurement conditions, as shown by the peak or geometric mean value of the distribution curves of the coated or control particles' diffusion coefficients (see, e.g., FIGS. 3 and 4, “PS Control” vs. “Control in Mucin”).

More specific steps of measurement and analysis the exemplary method to determine diffusion coefficients of coated/control particles are described in the Examples below.

The coated particles have a diffusion coefficient in mucus or a mucin solution (e.g. 3%-7% w/v mucin, such as 5% w/v mucin) that is at least 1.1-fold, at least 1.3-fold, at least 2-fold, at least 5-fold, at least 10-fold, at least 15-fold, at least 20-fold, in a range from 1.3-fold to 50-fold, from 1.3-fold to 40-fold, from 1.3-fold to 30-fold, from 1.3-fold to 25-fold, from 2-fold to 50-fold, from 2-fold to 40-fold, from 2-fold to 30-fold, from 2-fold to 25-fold, from 5-fold to 50-fold, from 5-fold to 40-fold, from 5-fold to 30-fold, or from 5-fold to 25-fold greater than the diffusion coefficient of control particles containing the core only in the same mucus or mucin solution, under the same measurement conditions, optionally using the selected region of interest method or the full image method described above.

Optionally, the coated particles show a reduced particle aggregation in mucus or a mucin solution (e.g. 3%-7% w/v mucin, such as 5% w/v mucin), as shown by a reduced number of clusters detected, by at least 5%, at least 10%, in a range from 5% to 50%, from 5% to 40%, from 10% to 50%, or from 20% to 50%, compared to the number of clusters detected for control particles in the same mucus or mucin solution, under the same measurement conditions.

In some forms, the coated particles have a diffusion coefficient in mucus or a mucin solution (e.g. 3%-7% w/v mucin, such as 5% w/v mucin) that is at least 1.1-fold, at least 1.3-fold, at least 2-fold, at least 5-fold, at least 10-fold, at least 15-fold, at least 20-fold, in a range from 1.3-fold to 50-fold, from 1.3-fold to 40-fold, from 1.3-fold to 30-fold, from 1.3-fold to 25-fold, from 2-fold to 50-fold, from 2-fold to 40-fold, from 2-fold to 30-fold, from 2-fold to 25-fold, from 5-fold to 50-fold, from 5-fold to 40-fold, from 5-fold to 30-fold, or from 5-fold to 25-fold greater than the diffusion coefficient of control particles containing the core only in the same mucus or mucin solution, under the same measurement conditions, optionally using the selected region of interest method or the full image method described above, and show a reduced particle aggregation in mucus or a mucin solution (e.g. 0.1% or 5% w/v mucin), as shown by a reduced number of clusters detected, by at least 5%, at least 10%, in a range from 5% to 50%, from 5% to 40%, from 10% to 50%, or from 20% to 50%, compared to the number of clusters detected for control particles in the same mucus or mucin solution, under the same measurement conditions. In some forms, the coated particles having the increased diffusion coefficient and/or reduced particle aggregation in mucus or a mucin solution (e.g. 3%-7% w/v mucin, such as 5% w/v mucin) as described above contain a core formed by polystyrene or PLA and a coating formed by any one of the glycoproteins described above.

In some forms, the coated particles have a diffusion coefficient in mucus or a mucin solution (e.g. 3%-7% w/v mucin, such as 5% w/v mucin) that is at least 1.1-fold, at least 1.3-fold, at least 2-fold, at least 5-fold, at least 10-fold, at least 20-fold, at least 30-fold, at least 40-fold, at least 50-fold, at least 100-fold, at least 150-fold, at least 160-fold, at least 170-fold, at least 200-fold, at least 250-fold, at least 300-fold, at least 500-fold, at least 1000-fold, at least 5000-fold, in a range from 1.3-fold to 10,000-fold, from 1.3-fold to 8000-fold, from 1.3-fold to 5000-fold, from 1.3-fold to 1000-fold, from 1.3-fold to 500-fold, from 1.3-fold to 200-fold, from 1.3-fold to 100-fold, from 1.3-fold to 30-fold, from 1.3-fold to 25-fold, from 2-fold to 30-fold, from 2-fold to 25-fold, from 5-fold to 30-fold, from 5-fold to 25-fold, from 100-fold to 10,000-fold, from 100-fold to 8000-fold, from 100-fold to 5000-fold, from 100-fold to 1000-fold, from 100-fold to 500-fold, from 150-fold to 10,000-fold, from 150-fold to 8000-fold, from 150-fold to 5000-fold, from 150-fold to 1000-fold, from 150-fold to 500-fold, or from 500-fold to 10,000-fold, greater than the diffusion coefficient of control particles containing the same core only in water, under the same measurement conditions, optionally using the selected region of interest method or the full image method described above.

In other words, the diffusion coefficient of the coated particles in mucus or a mucin solution (e.g. 3%-7% w/v mucin, such as 5% w/v mucin) has an enhancement factor of at least 1.1, at least 1.3, at least 2, at least 5, at least 10, at least 20, at least 30, at least 40, at least 50, at least 100, at least 150, at least 160, at least 170, at least 200, at least 250, at least 300, at least 500, at least 1000, at least 5000, in a range from 1.3 to 10,000, from 1.3 to 8000, from 1.3 to 5000, from 1.3 to 1000, from 1.3 to 500, from 1.3 to 200, from 1.3 to 100, from 1.3 to 30, from 1.3 to 25, from 2 to 30, from 2 to 25, from 5 to 30, from 5-fold to 25, from 100 to 10,000, from 100 to 8000, from 100 to 5000, from 100 to 1000, from 100 to 500, from 150 to 10,000, from 150 to 8000, from 150 to 5000, from 150 to 1000, from 150 to 500, or from 500 to 10,000, compared to the diffusion coefficient of control particles containing the same core only in water, under the same measurement conditions, optionally using the selected region of interest method or the full image method described above.

A first enhancement factor of the diffusion coefficient for coated particles in mucus or a mucin solution (e.g. 3%-7% w/v mucin, such as 5% w/v mucin) can be determined by comparing the diffusion coefficient of the coated particles in mucus or mucin solution to the diffusion coefficient of the control particles in water. For example, as shown in Example 1, Table 1, coated particles of poly(methyl methacrylate), polystyrene, and polylactic acid had a diffusion coefficient in 5% w/v mucin solution with a first enhancement factor in the range from greater than 1, such as about 1.3, to about 7800 compared to the diffusion coefficient of the respective control particles (i.e., poly(methyl methacrylate), polystyrene, or polylactic acid particles without any coating) in water. The enhancement factor for the diffusion coefficient of the coated particles can be determined under the same measurement conditions, optionally using the selected region of interest method or the full image method described above. For example, the enhancement factors for the coated particles of poly(methyl methacrylate), polystyrene, and polylactic acid shown in Table 1 were determined using the selected region of interest method.

A second enhancement factor of the diffusion coefficient for control particles in mucus or a mucin solution (e.g. 3%-7% w/v mucin, such as 5% w/v mucin) can be determined by comparing the diffusion coefficient of the control particles in mucus or mucin solution to the diffusion coefficient of the same control particles in water. For example, as shown in Example 1, Table 1, control particles of poly(methyl methacrylate), polystyrene, and polylactic acid had a diffusion coefficient in 5% mucin solution with an enhancement factor in the range from greater than 1, such as about 1.3, to about 370 compared to the diffusion coefficient of the same control particles in water. This can be determined under the same measurement conditions, optionally using the selected region of interest method or the full image method described above. For example, the enhancement factors for the control particles of poly(methyl methacrylate), polystyrene, and polylactic acid shown in Table 1 were determined using the selected region of interest method.

In some forms, the coated particles have a first enhancement factor that is greater than a second enhancement factor of the control particles (the control particles contain the same core as the coated particles, but without the coating). In these forms, the first enhancement factor is at least 1.1-fold, at least 1.5-fold, at least 2-fold, at least 3-fold, at least 5-fold, at least 6-fold, at least 7-fold, at least 8-fold, at least 10-fold, at least 12-fold, at least 15-fold, at least 20 fold, in a range from 1.1-fold to 100-fold, from 1.1-fold to 80-fold, from 1.1-fold to 60-fold, from 1.1-fold to 50-fold, from 1.1-fold to 40-fold, from 1.1-fold to 30-fold, from 2-fold to 100-fold, from 2-fold to 80-fold, from 2-fold to 60-fold, from 2-fold to 50-fold, from 2-fold to 40-fold, from 2-fold to 30-fold, from 5-fold to 100-fold, from 5-fold to 80-fold, from 5-fold to 60-fold, from 5-fold to 50-fold, from 5-fold to 40-fold, or from 5-fold to 30-fold, such as about 1.1-fold, about 5-fold, or about 20-fold, greater than the second enhancement factor. For example, as shown in Table 1, the coated particles of polystyrene have a first enhancement factor (i.e. 860.28); the control particles of polystyrene have a second enhancement factor (i.e. 172.06). Thus, the first enhancement factor of the coated particles of polystyrene is about 5 times (5-fold) greater than the second enhancement of the control particles of polystyrene. For example, as shown in Table 1, the coated particles of polylactic acid have a first enhancement factor (i.e. 7876.48); the control particles of polylactic acid have a second enhancement factor (i.e. 366.85). Thus, the first enhancement factor of the coated particles of polylactic acid is about 21 times (21-fold) greater than the second enhancement of the control particles of polylactic acid.

b. Systemic Uptake

Detecting coated particles containing active agents in the GI tract can be used to signify local delivery agents along segments of the GI tract. Methods for detecting coated particles in segments of the GI tract are known, such as by using FTIR as described in US 2021/0186880 by Mathiowitz, et al.

Detecting coated particles containing hydrophobic drugs and/or active agents in the blood can be used to signify successful absorption of both the coated particles and the hydrophobic drugs and/or active agents being delivered into systemic circulation. Any method known to those of skill in the art to determine polymers in samples (e.g. blood) can be used. Examples include gel permeation chromatography (GPC), high-performance liquid chromatography (HPLC), FTIR, mass spectrometry, and a combination of both (LC-MS).

When the coated particles are administered to a mammal, such as by oral administration or mucosal administration (such as local administration to a mucosal surface), uptake of the coated particles into the systemic circulation (i.e. systemic uptake) can reach an appreciable level, such as in a range from 10% to 90%, for example, about 30% in the blood of the mammal, as determined using Fourier Transform Infrared spectroscopy (“FTIR”). Specific methods for detecting and quantifying coated particles in blood using FTIR are described in US 2021/0186880 by Mathiowitz, et al. Control particles without the coating usually have a lower systemic absorption or uptake, such as <10%, compared to the coated particles described herein.

For example, when the coated particles are administered to a mammal, such as by oral administration or mucosal administration (local), systemic uptake of the coated particles can be in a range from 10% to 90%, from 15% to 85%, from 15% to 80%, from 15% to 70%, from 15% to 60%, from 15% to 50%, from 20% to 90%, from 20% to 80%, from 20% to 70%, from 20% to 60%, or from 20% to 50% in the mammal, such as about 30%, as measured using Fourier Transform Infrared spectroscopy.

c. Bioadhesivity

When the coated particles contain a polymeric core, the polymeric core may be formed by a bioadhesive polymer, such as polyesters (poly(caprolactone); poly(hydroxy acids), such as poly(lactic acid), poly(glycolic acid), and poly(lactic acid-co-glycolic acids); polyhydroxyalkanoates, such as poly(3-hydroxybutyrate) and poly(4-hydroxybutyrate)); polyanhydrides (poly(fumaric-co-sebacic acid), polysebacic acid, polyfumaric acid), thereby provides bioadhesivity for the coated particles. These bioadhesive polymers forming the core of the coated particle can have a bioadhesion force of about 100 mN/cm2 or greater, as measured in bulk using a texture analyzer and ex vivo tissue.

Without being bound to theory, it is believed that bioadhesivity of the polymeric core may help enhance systemic uptake of such coated particles by providing sufficient bioadhesion to prevent clearance and increase residence time, of the coated particles, in addition to the increased diffusion coefficient provided by the coating of the coated particles. Accordingly, in some forms, the coated particles containing polymeric cores have a near neutral zeta potential in a range from −16 mV to 16 mV, from −16 mV to 10 mV, from −16 mV to 5 mV, from −16 mV to 0 mV, from −16 mV to −5 mV, or from −16 mV to −10 mV, and a bioadhesion force of about 100 mN/cm² (such as 480 mN/cm²), or greater.

d. Hydrodynamic Diameter

The coated particles typically have an average hydrodynamic diameter in a range from 100 nm to 5000 nm, such as from 100 nm to 2000 nm, from 100 nm to 1000 nm, from 100 nm to 500 nm, from 500 nm to 1000 nm, from 500 nm to 2000 nm, or from 1000 nm to 2000 nm. The term “hydrodynamic diameter” refers to the diameter of a coated particle measured in an aqueous solution using dynamic light scattering or scanning electron microscopy. Based on whether delivery is systemic, such as to the systemic circulation, or local, such as to the GI tract or other mucosal surfaces (e.g. vagina) in a mammal, the coated particles can have different size sub-ranges within this range.

For example, while the coated particles penetrate tissue, they can also be taken up into systemic circulation (reaching the blood circulation). Thus, the coated particles can have hydrodynamic diameters depending on the location and/or polymer type/hydrophobic drug and/or type of delivery desired, namely local delivery in which an agent to be delivered predominantly remains in the GI tract or systemic delivery where the agent and/or hydrophobic drug in the coated particles are absorbed into systemic circulation.

Therefore, in some forms, the average hydrodynamic diameter of the coated particles for local delivery is in the range from 900 nm to 5000 nm, such as from 900 nm to 2000 nm. In some forms, the average hydrodynamic diameter of the coated particles for systemic delivery is in the range from 100 nm to 2000 nm, such as from 100 nm to 800 nm.

### B. Pharmaceutical Formulations

Pharmaceutical formulations that contain a plurality of the coated particles described herein in a form suitable for administration to a mammal, and particularly for delivery to a mucosal surface, such as the GI track and vagina, and/or blood circulation of the mammal, are disclosed. Typically, the hydrophobic drug(s) and/or active agent(s), when present, in the pharmaceutical formulation is present in an amount effective to prevent, treat, or ameliorate one or more symptoms associated with a disease or disorder of interest.

The pharmaceutical formulation may include one or more pharmaceutically acceptable carriers and/or one or more pharmaceutically acceptable excipients. For example, the pharmaceutical formulation may be in the form of a liquid, such as a solution or a suspension, and contain a plurality of the disclosed coated particles in an aqueous medium and, optionally, one or more suitable excipients for the liquid formulation. Optionally, the pharmaceutical formulation is in a solid form, and contains a plurality of coated particles and one or more suitable excipients for a solid formulation.

### 1. Carriers and Excipients

The pharmaceutical formulation contains one or more pharmaceutically acceptable carriers and/or excipients. Suitable pharmaceutically acceptable carriers and excipients are generally recognized as safe (GRAS), and may be administered to an individual without causing undesirable biological side effects or unwanted interactions.

Representative carriers and excipients include solvents (including buffers), diluents, pH modifying agents, preservatives, antioxidants, suspending agents, wetting agents, viscosity modifiers, tonicity agents, and stabilizing agents, and a combination thereof.

Coated particles for delivering active agents to the blood circulation or a mucosal surface of the mammal can be dissolved or suspended in a suitable carrier to form a liquid pharmaceutical formulation, such as sterile saline, phosphate buffered saline (PBS), balanced salt solution (BSS), viscous gel, or other pharmaceutically acceptable carriers for administration. The pharmaceutical formulation may also be a sterile solution, suspension, or emulsion in a nontoxic, parenterally acceptable diluent or solvent.

Excipients can be added to a liquid or solid pharmaceutical formulation to assist in sterility, stability (e.g. shelf-life), integration, and to adjust and/or maintain pH or isotonicity of the nanoparticles in the pharmaceutical formulation, such as diluents, pH modifying agents, preservatives, antioxidants, suspending agents, wetting agents, viscosity modifiers, tonicity agents, and stabilizing agents, and a combination thereof.

### 2. Form

The pharmaceutical formulation containing a plurality of the disclosed coated particles can be in a liquid form or a solid form, as a liquid formulation or a solid formulation for oral administration or mucosal administration (e.g. intravaginal administration, pulmonary administration, and/or administration to other mucosal surfaces) to a subject.

a. Oral Formulations

Optionally, the pharmaceutical formulation containing a plurality of the disclosed coated particles is in a form suitable for oral administration to a subject, such as a mammal (i.e. an oral fonnulation). Oral administration may involve swallowing, so that the coated particles containing active agent(s) enter the gastrointestinal tract.

Formulations suitable for oral administration include solid formulations such as tablets, capsules containing particulates, liquids, powders, lozenges (including liquid-filled lozenges), chews, multi- and nano-particulates, gels, solid solutions, liposomes, films, ovules, sprays, and liquid formulations.

Liquid formulations for oral administration include suspensions, solutions, syrups, and elixirs. Such oral formulations may be employed as fillers in soft or hard capsules and can contain one or more suitable carriers and/or excipients, for example, water, ethanol, polyethylene glycol, propylene glycol, chitosan polymers and chitosan derivatives (e.g. N-trimethylene chloride chitosan, chitosan esters, chitosan modified with hydrophilic groups, such as amino groups, carboxyl groups, sulfate groups, etc.), methylcellulose, a suitable oil, one or more emulsifying agents, and/or suspending agents. Liquid formulations for oral administration may also be prepared by the reconstitution of a solid, for example, from a sachet. Optionally, the coated particles are included in a fast-dissolving and/or fast-disintegrating dosage form.

For tablet or capsule dosage forms, in addition to the coated particles described herein, tablets generally contain disintegrants, binders, diluents, surface active agents, lubricants, glidants, antioxidants, colourants, flavouring agents, preservatives, or taste masking agents, or a combination thereof.

Examples of suitable disintegrants for forming a table or capsule dosage form containing the coated particles include, but are not limited to, sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant can have a concentration in a range from about 1 wt % to about 25 wt %, from about 5 wt % to about 20 wt % of the tablet or capsule dosage form containing the coated particles.

Binders are generally used to impart cohesive qualities to a tablet formulation. Suitable binders for forming a tablet or capsule formulation containing the coated particles include, but are not limited to, microcrystalline cellulose, gelatin, sugars, polyethylene glycol, natural and synthetic gums, polyvinylpyrrolidone, pregelatinised starch, chitosan polymers and chitosan derivatives (e.g. N-trimethylene chloride chitosan, chitosan esters, chitosan modified with hydrophilic groups, such as amino groups, carboxyl groups, sulfate groups, etc.), hydroxypropyl cellulose, and hydroxypropyl methylcellulose.

Suitable diluents for forming a table or capsule formulation containing the coated particles include, but are not limited to, lactose (as, for example, the monohydrate, spray-dried monohydrate or anhydrous form), chitosan polymers and chitosan derivatives (e.g. N-trimethylene chloride chitosan, chitosan esters, chitosan modified with hydrophilic groups, such as amino groups, carboxyl groups, sulfate groups, etc.), N-sulfonated derivatives of chitosan, quaternarized derivatives of chitosan, carbosyalkylated chitosan, microcrystalline chitosan, mannitol, xylitol, dextrose, sucrose, sorbitol, microcrystalline cellulose, starch and dibasic calcium phosphate dihydrate.

Tablet or capsule formulations containing the coated particles may also contain surface active agents, such as sodium lauryl sulfate and polysorbate 80, and glidants such as silicon dioxide and talc. When present, surface active agents can have a concentration in a range from about 0.2 wt % to 5 wt % of the tablet or capsule formulation.

Tablet or capsule formulations containing the coated particles also generally contain lubricants, such as magnesium stearate, calcium stearate, zinc stearate, sodium stearyl fumarate, and mixtures of magnesium stearate with sodium lauryl sulphate. Lubricants can have a concentration in a range from about 0.25 wt % to 10 wt %, from about 0.5 wt % to about 3 wt % of the tablet or capsule formulation.

Other possible excipients included in a tablet or capsule formulation containing the coated particles include glidants (e.g. Talc or colloidal anhydrous silica at about 0.1 wt % to about 3 wt % of the table or capsule formulation), antioxidants, colourants, flavouring agents, preservatives and taste-masking agents. When present, glidants can have a concentration in a range from about 0.2 wt % to 1 wt % of the tablet or capsule formulation.

An exemplary tablet formulation contains up to about 80 wt % of the coated particles described herein, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet or capsule blends, including the coated particles and one or more suitable excipients, may be compressed directly or by roller to form tablets. Tablet or capsule blends or portions of the blends may alternatively be wet-, dry-, or melt-granulated, melt congealed, or extruded before tableting. The final table or capsule formulation may contain one or more layers and may be coated or uncoated; it may even be encapsulated in another large particle, such as a liposomal particle.

Solid formulations containing the coated particles for oral administration may be formulated to be immediate and/or modified release. Modified release formulations include delayed, sustained, pulsed, controlled, targeted and programmed release formulations.

b. Mucosal Formulations

The coated particles can be formulated for pulmonary or mucosal administration. The administration can include delivery of the composition to the lungs, nasal, oral (sublingual, buccal), vaginal, or rectal mucosa.

For example, the coated particles can also be administered intranasally or by oral inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as water, ethanol-water mixture, 1,1,1,2-tetrafluoroethane or 1,1,1,2,3,3,3-heptafluoropropane. For intranasal or oral inhalation use, the powder may contain a bioadhesive agent, for example, chitosan or cyclodextrin. The term aerosol as used herein refers to any preparation of a fine mist of particles, which can be in solution or a suspension, whether or not it is produced using a propellant. Aerosols can be produced using standard techniques, such as ultrasonication or high-pressure treatment.

The pressurized container, pump, spray, atomizer, or nebulizer contains a solution or suspension of one or more of the compounds including, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Capsules (made, for example, from gelatin or hydroxypropylmethylcellulose), blisters and cartridges for use in an inhaler or insufflator may be formulated to contain a powder mix of the coated particles described herein, a suitable powder base such as lactose or starch and a performance modifier such as 1-leucine, mannitol, or magnesium stearate. The lactose may be anhydrous or in the form of a monohydrate, preferably the latter. Other suitable excipients include dextran, glucose, maltose, sorbitol, xylitol, fructose, sucrose, and trehalose.

A suitable solution formulation for use in an atomizer using electrohydrodynamics to produce a fine mist may contain from 1 μg to 20 mg of one or more of the coated particles per actuation and the actuation volume may vary from 1 μl to 100 μl. A typical formulation may contain a plurality of the coated particles disclosed herein, propylene glycol, sterile water, ethanol and sodium chloride. Alternative solvents that may be used instead of propylene glycol include glycerol and polyethylene glycol.

Suitable flavors, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using for example, PGLA. Modified release formulations include delayed, sustained, pulsed, controlled, targeted, and programmed release formulations.

In the case of dry powder inhalers and aerosols, the dosage unit is determined by means of a valve which delivers a metered amount. Units in accordance with the coated particles are typically arranged to administer a metered dose or “puff”. The overall daily dose will be administered in a single dose or, more usually, as divided doses throughout the day.

In some forms, the coated particles can be formulated for pulmonary delivery, such as intranasal administration or oral inhalation. Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into an aqueous solution, e.g., water or isotonic saline, buffered or un-buffered, or as an aqueous suspension, for intranasal administration as drops or as a spray. Such aqueous solutions or suspensions may be isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

In some forms, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In some forms, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the fornulation. The solvent should not detrimentally react with the coated particles. An appropriate solvent should be used that forms a suspension of the coated particles. The solvent should be sufficiently volatile to enable formation of an aerosol of the solution or suspension. Additional solvents or aerosolizing agents, such as freons, can be added as desired to increase the volatility of the solution or suspension.

In some forms, the pharmaceutical formulations may contain minor amounts of surfactants or other excipients well known to those of the art. In this context, “minor amounts” means no excipients are present that might affect or mediate penetration of the coated particles in tissues and that the excipients that are present in amount that do not adversely affect penetration of the coated particles in tissues.

In some forms, the pharmaceutical formulations containing a plurality of the coated particles can be administered directly to the mucous membranes (including the surface membranes of the nose, lungs and mouth), such that the coated particles cross the mucosal layer and enters the underlying tissues.

Such formulations for direct application on the mucous membrane generally contain a dermatologically acceptable carrier that is suitable for application to the mucous membrane, has good aesthetic properties, is compatible with the active agents and any other components, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse or a transdermal patch. Other suitable topical carriers include anhydrous liquid solvents such as oils, alcohols, and silicones (e.g., mineral oil, ethanol isopropanol, dimethicone, cyclomethicone, and the like); aqueous-based single phase liquid solvents (e.g., hydro-alcoholic solvent systems, such as a mixture of ethanol and/or isopropanol and water); and thickened versions of these anhydrous and aqueous-based single phase solvents (e.g. where the viscosity of the solvent has been increased to form a solid or semi-solid by the addition of appropriate gums, resins, waxes, polymers, salts, and the like). Examples of topical carrier systems useful in the present formulations are described in the following four references all of which are incorporated herein by reference in their entirety: “Sun Products Formulary” Cosmetics & Toiletries, vol. 105, pp. 122-139 (December 1990); “Sun Products Formulary,” Cosmetics & Toiletries, vol. 102, pp. 117-136 (March 1987); U.S. Pat. No. 5,605,894 to Blank et al., and U.S. Pat. No. 5,681,852 to Bissett.

Formulations for direct application on the mucous membrane may be formulated to be immediate and/or modified release. Modified release formulations include delayed, sustained, pulsed, controlled, targeted and programmed release formulations. Thus, the coated particles may be formulated as a solid, semi-solid, or thixotropic liquid for administration as an implanted depot providing modified release of the active agents. Examples of such formulations include drug-coated stents.

### 3. Effective Amount

The pharmaceutical formulation contains an effective amount of the hydrophobic drug(s) and active agent(s), when present, encapsulated in a polymeric core and/or embedded in the coating of the coated particles for preventing, treating, or ameliorating one or more symptoms of a given disease or disorder. As used herein, the terms “effective amount” and “therapeutically effective amount” mean a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

Generally, when the coated particle contains one or more hydrophobic drug(s) as the core, the total concentration of the hydrophobic drug(s) in the pharmaceutical formulation is in a range from about 0.001 wt % to about 20 wt %, from about 0.001 wt % to about 15 wt %, from about 0.001 wt % to about 10 wt %, from about 0.001 wt % to about 5 wt %, from about 0.001 wt % to about 2 wt %, from about 0.001 wt % to about 0.5 wt %, from about 0.001 wt % to about 0.2 wt %, from about 0.001 wt % to about 0.1 wt %, from about 0.01 wt % to about 20 wt %, from about 0.01 wt % to about 15 wt %, from about 0.01 wt % to about 10 wt %, from about 0.01 wt % to about 5 wt %, from about 0.01 wt % to about 2 wt %, from about 0.01 wt % to about 0.5 wt %, from about 0.01 wt % to about 0.2 wt %, from about 0.01 wt % to about 0.1 wt %, from about 0.1 wt % to about 20 wt %, from about 0.1 wt % to about 15 wt %, from about 0.1 wt % to about 10 wt %, from about 0.1 wt % to about 5 wt %, from about 0.1 wt % to about 2 wt %, from about 0.1 wt % to about 0.5 wt %, or from about 0.1 wt % to about 0.2 wt %. The term “total concentration of the hydrophobic drug(s) in the pharmaceutical formulation” refers to the sum of the weight of all hydrophobic drug(s) of the coated particle relative to the weight of the formulation.

When the coated particle containing one or more hydrophobic drug(s) as the core and one or more additional active agent(s) embedded in the coating of the coated particles, the total concentration of the active agent(s) in the pharmaceutical formulation is in a range from about 0.001 wt % to about 20 wt %, from about 0.001 wt % to about 15 wt %, from about 0.001 wt % to about 10 wt %, from about 0.001 wt % to about 5 wt %, from about 0.001 wt % to about 2 wt %, from about 0.001 wt % to about 0.5 wt %, from about 0.001 wt % to about 0.2 wt %, from about 0.001 wt % to about 0.1 wt %, from about 0.01 wt % to about 20 wt %, from about 0.01 wt % to about 15 wt %, from about 0.01 wt % to about 10 wt %, from about 0.01 wt % to about 5 wt %, from about 0.01 wt % to about 2 wt %, from about 0.01 wt % to about 0.5 wt %, from about 0.01 wt % to about 0.2 wt %, from about 0.01 wt % to about 0.1 wt %, from about 0.1 wt % to about 20 wt %, from about 0.1 wt % to about 15 wt %, from about 0.1 wt % to about 10 wt %, from about 0.1 wt % to about 5 wt %, from about 0.1 wt % to about 2 wt %, from about 0.1 wt % to about 0.5 wt %, or from about 0.1 wt % to about 0.2 wt %. The term “total concentration of the active agent(s) in the pharmaceutical formulation” refers to the sum of the weight of all active agent(s) embedded in the coating of the coated particle relative to the weight of the formulation.

When the coated particle contains a polymeric core (i.e. coated polymeric particle), the coated particles contain one or more active agent(s) encapsulated in the polymeric core and/or embedded in the coating of the coated particles. In such coated particles, the total concentration of the active agent(s) in the pharmaceutical formulation is in a range from about 0.001 wt % to about 20 wt %, from about 0.001 wt % to about 15 wt %, from about 0.001 wt % to about 10 wt %, from about 0.001 wt % to about 5 wt %, from about 0.001 wt % to about 2 wt %, from about 0.001 wt % to about 0.5 wt %, from about 0.001 wt % to about 0.2 wt %, from about 0.001 wt % to about 0.1 wt %, from about 0.01 wt % to about 20 wt %, from about 0.01 wt % to about 15 wt %, from about 0.01 wt % to about 10 wt %, from about 0.01 wt % to about 5 wt %, from about 0.01 wt % to about 2 wt %, from about 0.01 wt % to about 0.5 wt %, from about 0.01 wt % to about 0.2 wt %, from about 0.01 wt % to about 0.1 wt %, from about 0.1 wt % to about 20 wt %, from about 0.1 wt % to about 15 wt %, from about 0.1 wt % to about 10 wt %, from about 0.1 wt % to about 5 wt %, from about 0.1 wt % to about 2 wt %, from about 0.1 wt % to about 0.5 wt %, or from about 0.1 wt % to about 0.2 wt %. The term “total concentration of the active agent(s) in the pharmaceutical formulation” refers to the sum of the weight of all active agent(s) encapsulated in the core and/or embedded in the coating of the coated polymeric particle relative to the weight of the formulation.

### II. Methods of Making Coated Particles

When the coated particle contains a polymeric core, the polymeric core can be manufactured using any suitable known method, such as solvent evaporation (“SE”) or phase inversion nanoencapsulation (“PIN”). SE is described in detail in Mathiowitz, et al., J. Appl. Polym. Sci. 35:755-774 (1988), the contents of which are incorporated herein by reference. U.S. Patent Application Publication US20040070093A1 by Mathiowitz, et al. and U.S. Pat. No. 6,123,965 to Jacob and Mathiowitz, describe phase inversion nanoencapsulation, the contents of which are incorporated herein by

### REFERENCE

Briefly, phase inversion is a physical process in which a polymer is first dissolved in “good” solvent, forming one continuous homogenous liquid phase. By adding this mixture to the excess of a non-solvent (or “bad” solvent), an unstable two- phase mixture of polymer rich and polymer poor fractions is formed, causing the polymer to aggregate at the nucleation points. When the polymer concentration reaches a certain point (cloud point), polymeric cores phase separate, solidifying and precipitating from the solution.

Unlike solvent removal or solvent evaporation methods, PIN does not require emulsification of the initial continuous phase polymer/solvent solution. It utilizes low polymer concentrations and low viscosities of the encapsulants. Also, the solvent and non-solvent pairs are preferably miscible with at least ten times excess of non-solvent relative to solvent. These conditions allow for rapid addition of polymer dissolved in continuous solvent phase into non-solvent, which in turn result in spontaneous formation of nanomaterial or micromaterial. Since no emulsification is required in this process and the nanospheres or microspheres form spontaneously, the size of the resulting spheres is controlled not by the speed of stirring, but rather by changing the parameters of the procedure: polymer concentration, solvent to non-solvent ratio and their miscibility.

The coating of the coated particles can be formed by (i) dispersing a polymeric core or hydrophobic drug in a solution containing a glycoprotein or a combination of sugar, oligosaccharide, and/or polysaccharide, and a protein, to form a suspension and maintain it for a time period sufficient to form a coating on the polymeric core or hydrophobic drug; and optionally (ii) drying the suspension by lyophilization or vacuum oven. Optionally, the coated particles are encapsulated in an enteric polymeric capsule, which dissolves at a suitable pH depending on the type of delivery desired.

Any known method of incorporating an active agent into or onto a polymeric core and/or coating of the coated particles can be used to incorporate a non-biological agent. Suitable methods include, but are not limited to solvent evaporation (e.g. emulsion and solvent evaporation), nanoprecipitation, microfluidics, self-assembly, solvent diffusion/displacement, solvent removal, spray drying, etc. Optionally, for biological agents (e.g. proteins), solvent evaporation and/or PIN are used to incorporate the active agents into or onto the coated particles while retaining the activities of the biological agents.

### III. Methods of Using

### A. Treating Diseases or Disorders

The pharmaceutical formulations containing a plurality of the coated particles disclosed herein can be administered to a subject in need thereof, for the treatment of a variety of diseases or disorders.

Generally, the method includes administering to the subject the pharmaceutical formulation containing a plurality of the coated particles, wherein the administration step occurs one or more times.

Methods of administration of the formulations can be oral, i.e., administration to or by way of the mouth, to provide uptake through the GI tract; or mucosal, such as intranasally, inhalation, or intravaginal administration, or direct application to a mucous membrane in the subject. In systemic circulation, the coated particles may accumulate in diseased site.

In some forms, the pharmaceutical formation contains an effective amount of a hydrophobic drug forming the core of the coated particle to be delivered for preventing, treating, and/or ameliorating one or more symptoms of a given disease or disorder. In these forms, the pharmaceutical formulation may contain one or more additional active agent(s) embedded in the coating of the coated particle. In some forms, the pharmaceutical formulation contains a polymeric core and one or more active agent(s) to be delivered for preventing, treating, and/or ameliorating one or more symptoms of a given disease or disorder. In these forms, the one or more active agent(s) are encapsulated in the polymeric core and/or embedded in the coating of the coated particle.

The pharmaceutical formulations can be administered in a single dose or in multiple doses. Certain factors may influence the dosage required to effectively prevent, treat, or ameliorate the symptoms of a disease or disorder, including, but not limited to, the severity of the disease or disorder, previous preventions, the general health and/or age of the subject, and other diseases present. It will also be appreciated that the effective dosage of the hydrophobic drug(s) and/or active agent(s) used for prevention or treatment may increase or decrease over the course of particular prevention or treatment. Changes in dosage may result and become apparent from the results of assays.

Preventing a disease or disorder or the symptoms of the disease or disorder includes administering a pharmaceutical formulation containing the coated particles to a subject at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, or stabilization or delay of the development or progression of the disease or disorder, or to have a combination of these effects.

1. Diseases or Disorders being Treated

The pharmaceutical formulations described herein can be administered to a subject to prevent or treat any disease or disorder or ameliorate one or more symptoms associated with a disease or disorder.

The subject or patient is an individual who is the target of treatment using the disclosed formulations containing a plurality of the coated particles. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can also include a control subject or a test subject.

Diseases or disorders that can be treated using the methods described herein include, but are not limited to, diabetes; autoimmune disorders (e.g. Crohn's disease, chronic arthritis, multiple sclerosis, Sjogren's disease, Lupus erythematosus, psoriasis, Celiac disease, etc); cancer (breast cancer (e.g., metastatic or locally advanced breast cancer), prostate cancer (e.g., hormone refractory prostate cancer), renal cell carcinoma, lung cancer (e.g., small cell lung cancer and non-small cell lung cancer (including adenocarcinoma, squamous cell carcinoma, bronchoalveolar carcinoma and large cell carcinoma)), pancreatic cancer, gastric cancer (e.g., gastroesophageal, upper gastric or lower gastric cancer), colorectal cancer, squamous cell cancer of the head and neck, ovarian cancer (e.g., advanced ovarian cancer, platinum-based agent resistant or relapsed ovarian cancer), lymphoma (e.g., Burkitt's, Hodgkin's or non-Hodgkin's lymphoma), leukemia (e.g., acute myeloid leukemia) and gastrointestinal cancer); pain; fungal infections; bacterial infections; inflammation; anxiety; etc.

The disclosed coated particles, pharmaceutical formulations, and methods can be further understood through the following numbered paragraphs.

1. Coated particles comprising:
a core comprising a polymer or a hydrophobic drug; and
a coating comprising a glycoprotein or a combination of a sugar and a protein,
wherein the glycoprotein or the combination of sugar and protein surrounds the core.
   2. The coated particles of paragraph 1, having an average hydrodynamic diameter in a range from 100 nm to 5000 nm, and optionally in a range from 100 nm to 2000 nm.
   3. The coated particles of paragraph 1 or 2, wherein the core comprises a hydrophobic drug.
   4. The coated particles of paragraph 1 or 2, wherein the core comprises a polymer and the polymer is biodegradable and biocompatible.
   5. The coated particles of paragraph 4, wherein the polymer is hydrophobic, hydrophilic, or amphiphilic.
   6. The coated particles of paragraph 4 or 5, wherein the polymer has a molecular weight in a range from 1.5 kDa to 300 kDa, from 1.5 kDa to 275 kDa, from 1.5 kDa to 250 kDa, from 1.5 kDa to 100 kDa, from 2 kDa to 80 kDa, from 2 kDa to 50 kDa, from 2 kDa to 30 kDa, from 2 kDa to 20 kDa, or from 2 kDa to 10 kDa.
   7. The coated particles of any one of paragraphs 4-6, wherein the polymer is selected from the group consisting of polystyrene, hydrogels, such as fibrin, collagen, gelatin, hyaluronic acid, alginate, cellulose, dextran, and agarose, poly(alkylene glycol), such as poly(ethylene glycol), poly(vinyl alcohol), poly(acrylic acid), polyacrylate, such as poly(methyl methacrylate), poly(2-hydroxyethyl methacrylate), poly(ethyleneglycolmethacrylate), poly(oligoethylene glycol methacrylate), poly(ethyleneglycol dimethacrylate), and poly(diacrylate), polyacrylamide, such as poly(isopropylacrylamide), poly(vinyl pyrrolidone), hydrophobic peptides, polyesters, such as poly(caprolactone), poly(hydroxyacids), such as poly(lactic acid), poly(glycolic acid), and poly(lactic acid-co-glycolic acid), polyhydroxyalkanoates, such as poly(3-hydroxybutyrate) and poly(4-hydroxybutyrate), polyanhydrides, such as poly(fumaric-co-sebacic acid), poly[butadiene-co-(maleic anhydride)], polysebacic acid, polyfumaric acid, poly(orthoesters), hydrophobic polypeptides, hydrophobic polyethers, such as poly(propylene oxide), poly(phosphazenes), polyesteramides, poly(alkylene alkylates), polyether esters, polyacetals, polycyanoacrylates, polyketals, polyhydroxyvalerates, polyalkylene oxalates, polyalkylene succinates, mixtures, and copolymers thereof.
   8. The coated particles of any one of paragraphs 4-7, wherein the polymer is selected from the group consisting of polystyrene, poly(methyl methacrylate), poly(ethylene glycol), poly(lactic acid), poly[butadiene-co-(maleic anhydride)], poly(fumaric-co-sebacic acid), poly(glycolic acid), poly(lactic acid-co-glycolic acid), polysebacic acid, polyfumaric acid, mixtures, and copolymers thereof.
   9. The coated particles of any one of paragraphs 4-8, wherein the polymer is poly(lactic acid) or poly(lactic acid-co-glycolic acid), or a combination thereof.
   10. The coated particles of any one of paragraphs 4-9, wherein the polymer is bioadhesive, optionally wherein the polymer has a bioadhesion force of about 100 mN/cm² or greater, or about 500 mN/cm² or greater.
   11. The coated particles of any one of paragraphs 1-3, wherein the hydrophobic drug is a Biopharmaceutical Classification System (“BCS”) Class IV drug.
   12. The coated particles of any one of paragraphs 1-11, wherein the coating comprises a glycoprotein selected from the group consisting of collagen, mucins, transferrin, ceruloplasmin, immunoglobulins, histocompatibility antigens, human chorionic gonadotropin, thyroid-stimulating hormone, enzymes, such as alkaline phosphatase and patatin, proteins involved in cell-cell, virus-cell, bacterium-cell, and/or hormone-cell interactions, plasma proteins of coldwater fish, lectins, selectins, antibodies that interact with carbohydrates, proteins involved in hormone and drug actions, calnexin, calreticulin, notch and its analogs, proteins involved in the regulation of development, glycoproteins on the surface membranes of platelets.
   13. The coated particles of any one of paragraphs 1-12, wherein the coating comprises a combination of a sugar, an oligosaccharide, and/or a polysaccharide, and a protein, and wherein the sugar, oligosaccharide, and/or polysaccharide is covalently or non-covalently bound to the protein.
   14. The coated particles of paragraph 13, wherein the sugar, oligosaccharide, and/or polysaccharide is/are selected from the group consisting of sucrose, glucose, fucose, mannose, galactose, xylose, an oligosaccharide thereof, and a polysaccharide thereof, and wherein the protein contains an amino acid bone comprising at least 50 repeated units of serine and threonine.
   15. The coated particles of any one of paragraphs 1-14, wherein the coating further comprises one or more excipients, and optionally wherein the total concentration of the excipients in the coating is less than 50% (wt), in a range from about 0.01% (wt) to about 45% (wt), from about 0.05% (wt) to about 45% (wt), from about 0.1% (wt) to about 45% (wt), from about 0.01% (wt) to about 40% (wt), from about 0.05% (wt) to about 40% (wt), from about 0.1% (wt) to about 40% (wt), from about 0.01% (wt) to about 30% (wt), from about 0.05% (wt) to about 30% (wt), from about 0.1% (wt) to about 30% (wt), from about 0.01% (wt) to about 20% (wt), from about 0.05% (wt) to about 20% (wt), from about 0.1% (wt) to about 20% (wt), from about 0.01% (wt) to about 10% (wt), from about 0.05% (wt) to about 10% (wt), from about 0.1% (wt) to about 10% (wt), from about 0.01% (wt) to about 5% (wt), from about 0.05% (wt) to about 5% (wt), from about 0.1% (wt) to about 5% (wt), from about 0.01% (wt) to about 1% (wt), from about 0.05% (wt) to about 1% (wt), or from about 0.1% (wt) to about 1% (wt).
   16. The coated particles of any one of paragraphs 1-15 further comprising one or more active agents.
   17. The coated particles of paragraph 16, wherein the active agent is encapsulated in the core and/or embedded in the coating, or a combination thereof.
   18. The coated particles of paragraph 16 or 17, wherein the active agent is selected from the group consisting of small molecules, proteins, polypeptides, peptides, carbohydrates, nucleic acids, glycoproteins, lipids, antibodies/antigens, and combinations thereof, and wherein when the active agent is a protein or glycoprotein, the protein or glycoprotein is different from the glycoprotein or the protein forming the coating.
   19. The coated particles of any one of paragraphs 1-3 and 11-18, wherein the weight percentage of the hydrophobic drug in the coated particles is in a range from 0.01% to 10%, from 0.05% to 10%, from 0.1% to 10%, from 0.01% to 5%, from 0.05% to 5%, from 0.1% to 5%, from 0.01% to 2%, from 0.05% to 2%, from 0.1% to 2%, from 0.01% to 1%, from 0.05% to 1%, or from 0.1% to 1%.
   20. The coated particles of any one of paragraphs 16-19, wherein the weight percentage of the active agent in the coated particles is in a range from 0.01% to 10%, from 0.05% to 10%, from 0.1% to 10%, from 0.01% to 5%, from 0.05% to 5%, from 0.1% to 5%, from 0.01% to 2%, from 0.05% to 2%, from 0.1% to 2%, from 0.01% to 1%, from 0.05% to 1%, or from 0.1% to 1%.
   21. The coated particles of any one of paragraphs 1-20, wherein the coated particles have a diffusion coefficient in a mucin solution (3 to 7% wt) that is at least 1.1-fold, at least 1.3-fold, at least 2-fold, at least 5-fold, in a range from 1.3-fold to 30-fold, from 1.3-fold to 25-fold, from 2-fold to 30-fold, from 2-fold to 25-fold, from 5-fold to 30-fold, or from 5-fold to 25-fold greater than the diffusion coefficient of control particles consisting of the core in the same mucin solution, under the same measurement conditions.
   22. The coated particles of any one of paragraphs 1-21, wherein the coated particles have a diffusion coefficient in a mucin solution that is least 1.1-fold, at least 1.3-fold, at least 2-fold, at least 5-fold, at least 10-fold, at least 20-fold, at least 30-fold, at least 40-fold, at least 50-fold, at least 100-fold, at least 150-fold, at least 160-fold, at least 170-fold, at least 200-fold, at least 250-fold, at least 300-fold, at least 500-fold, at least 1000-fold, at least 5000-fold, in a range from 1.3-fold to 10,000-fold, from 1.3-fold to 8000-fold, from 1.3-fold to 5000-fold, from 1.3-fold to 1000-fold, from 1.3-fold to 500-fold, from 1.3-fold to 200-fold, from 1.3-fold to 100-fold, from 1.3-fold to 30-fold, from 1.3-fold to 25-fold, from 2-fold to 30-fold, from 2-fold to 25-fold, from 5-fold to 30-fold, from 5-fold to 25-fold, from 100-fold to 10,000-fold, from 100-fold to 8000-fold, from 100-fold to 5000-fold, from 100-fold to 1000-fold, from 100-fold to 500-fold, from 150-fold to 10,000-fold, from 150-fold to 8000-fold, from 150-fold to 5000-fold, from 150-fold to 1000-fold, from 150-fold to 500-fold, or from 500-fold to 10,000-fold, greater than the diffusion coefficient of control particles consisting of the core only in water, under the same measurement conditions.
   23. The coated particles of paragraphs 1-22, wherein the diffusion coefficient of the coated particles in a mucin solution has a first enhancement factor compared to the diffusion coefficient of control particles in water, under the same measurement conditions, wherein the diffusion coefficient of the control particles in the same mucin solution has a second enhancement factor compared to the diffusion coefficient of the control particles in water, under the same measurement conditions, and wherein the first enhancement factor is at least 1.1-fold, at least 1.5-fold, at least 2-fold, at least 3-fold, at least 5-fold, at least 6-fold, at least 7-fold, at least 8-fold, at least 10-fold, at least 12-fold, at least 15-fold, at least 20 fold, in a range from 1.1-fold to 100-fold, from 1.1-fold to 80-fold, from 1.1-fold to 60-fold, from 1.1-fold to 50-fold, from 1.1-fold to 40-fold, from 1.1-fold to 30-fold, from 2-fold to 100-fold, from 2-fold to 80-fold, from 2-fold to 60-fold, from 2-fold to 50-fold, from 2-fold to 40-fold, from 2-fold to 30-fold, from 5-fold to 100-fold, from 5-fold to 80-fold, from 5-fold to 60-fold, from 5-fold to 50-fold, from 5-fold to 40-fold, or from 5-fold to 30-fold, such as about 1.1-fold, about 5-fold, or about 20-fold, greater than the second enhancement factor.
   24. The coated particles of paragraphs 1-23, showing a reduced number of clusters detected in a mucin solution by at least 5%, at least 10%, in a range from 5% to 50%, from 5% to 40%, from 10% to 50%, or from 20% to 50%, compared to the number of clusters detected for control particles in the same mucin solution, under the same measurement conditions.
   25. The coated particles of any one of paragraphs 1-24, wherein when the coated particles are administered to a mammal, the systemic uptake is in a range from 10% to 90%, from 15% to 85%, from 15% to 80%, from 15% to 70%, from 15% to 60%, from 15% to 50%, from 20% to 90%, from 20% to 80%, from 20% to 70%, from 20% to 60%, or from 20% to 50% in a mammal, as measured using Fourier Transform Infrared spectroscopy.
   26. The coated particles of any one of paragraphs 1-25, wherein the core and the coating are not or do not contain polyethylene glycol.
   27. The coated particles of any one of paragraphs 1-26, wherein when the core is a polymer core, the coating is not mucin or does not contain mucin; the coating is not or does not contain mucin at a density that imparts a zeta potential in a range from −16 mV to −7.5 mV to the coated particles; or the coating is not formed by dispersing the core in a 0.1% (w/v) mucin solution.
   28. A pharmaceutical formulation comprising the coated particles of any one of paragraphs 1-27, and a pharmaceutically acceptable carrier and/or excipient.
   29. The pharmaceutical formulation of paragraph 28 in a form suitable for oral administration or intravaginal administration.
   30. A method for treating a disease or disorder in a subject in need thereof comprising:
(i) administering to the subject the pharmaceutical formulation of paragraph 28 or 29, wherein step (i) occurs one or more times.
   31. The method of paragraph 30, wherein the subject is a mammal.
   32. The method of paragraph 30 or 31, wherein in step (i) the pharmaceutical formulation is administered by oral administration or intravaginal administration, or a combination thereof.

“About,” as relates to numerical values described herein, refers to a value that is ±10% of the specified value.

Those skilled in the art will recognize, or be able to ascertain using no more than routine experimentation, many equivalents to the specific embodiments of the invention described herein. Such equivalents are intended to be encompassed by the ensuing claims.

The present invention will be further understood by reference to the following non-limiting examples.

### EXAMPLES

### Example 1. Nanoparticles with Coatings Show Increased Dₘ and Reduced Aggregation in Mucus Compared to the Same Nanoparticles without Coatings (Control Particles)

### 1 MATERIALS AND METHODS

### 1.1 Materials and Instrument

Polylactic acid (PLA, 8 kDa), poly lactic-co-glycolic acid (PLGA) (50:50) 6 and 10 kDa, PLGA (65:35) 6 kDa, and PLGA-ester end group (75:25 (monomer weight ratio), 8 kDa) were donated by Takeda Pharmaceuticals. Polystyrene (PS) 2.5, 45, and 125-250 kDa, PMMA (75 kDa), PLA 18 kDa, polychromatic red PS (PCRPS, 540 nm diameter), polyethylene glycol (PEG)-PLGA, and PS NPs (50-2000 nm) were purchased from Polysciences Inc. Poly fumaric acid sebacic acid (PFA:SA) 20:80 (14.5 kDa) and PBMAD (15-18 kDa) were synthesized as described in Sarosiek, et al., 1984, Witas, et al., 1983, and Lichtenberger, 1995. Dichloromethane (DCM), petroleum ether (PE), ethanol, polytetrafluoroethylene (PTFE) membrane (0.22 μm pore size, by Flouropore™), Drierite (8 mesh), heparin, KBr, span-80, polyvinyl alcohol (PVA), polydimethylsiloxane (PDMS), phosphate-buffered saline (PBS) were purchased from Fisher-Scientific. Mucin from porcine stomach Type II was purchased from Sigma-Aldrich and simulated intestinal fluid (SIF) without pancreatin, USP XXII formulation (RICCA chemicals, USA). The following materials were used for the TEM PS experiments and were purchased from Electron Microscopy Sciences: 20% paraformaldehyde, 25% glutaraldehyde, LR White™ (LRW), sodium dimethyl arsenate (C₂H₆AsNaO₂), osmium tetroxide (OsO₄), and uranyl acetate (UO₂(CH₃COO)₂).

### 1.2 Preparation of Polymeric NPs

Polymeric NPs were prepared via the Phase Inversion Nano-encapsulation (PIN) method (see U.S. Pat. No. 6,616,869 to Mathiowitz, et al.) to produce small NPs (<400 nm) and via solvent evaporation (SE) method (Jung, et al., 2000) to produce bigger NPs (>1000 nm). Briefly, in the PIN method, 1.5% (w/v) of polymers were dissolved in DCM (in ethanol for PBMAD). Next, the DCM solution was added to PE (1:100, respectively). NPs were filtered using a PTFE filter (average yield >75%). In the solvent evaporation (SE) method, 3 g of PS was dissolved in 15 mL of DCM with 0.1% span-80. Then it was added drop-wisely to a 250 mL solution of 1% PVA and 0.75 mL of Octanol while mixed at 10,000 RPM for 1 min. The mixture was then added to an additional 250 mL of 0.5% (w/v) PVA while stirred at 2000 RPM for 60 min. Finally, PS microparticles were washed with DI water and collected via centrifugation.

### 1.3 Diffusion Coefficient Measurements

### Selected Region of Interest Method:

(1) Dispersing polymeric cores in water or in 0.1% (w/v) mucin solution: about 0.2 mg of fluorescent polymeric cores (polymeric core encapsulating a fluorescent dye) were dispersed in 1 mL deionized water (“control particles”) or 0.1% mucin solution (“coated particles”), the resulting dispersions each had a concentration of 0.02% w/v. The dispersion was achieved by multiple vortexing and bath sonication (40 kHz), until floating or aggregates were no longer visually observed in the solution. This process generally takes no more than 15 minutes.

(2) Preparing a 5% mucin solution: a 5% (w/v) of mucin solution in DI was prepared by dissolving 50 mg of mucin in 1 mL DI water using vigorous stirring and/or occasional bath sonication at least two hours prior to measurements.

(3) Brownian motion measurement: 10⁻²⁰ μL of the dispersion (either control particles or coated particles) were added to about 200 μL of 5% mucin solution on a microscope slide, and covered and sealed, such as with nail polish, to prevent dehydration and convection. The solution was left for at least 5 minutes to allow full dispersion and avoid gradients. Before measurement, homogeneity in the solution was typically confirmed via scanning the entire slide to see if there was any non-random movement (i.e. in a specific direction). After homogeneity was confirmed (≤2 non-random movement), the sample was maintained for 5 minutes before measurement. For measuring the Brownian motion of the control particles or coated particles, laser intensity was fixed and background was corrected (to account for released/solute fluorescent molecules), and then time-lapse confocal images were captured in at least 10 different locations (typically a minimum of 600 NPs per measurement).

(4) Analyzing images: Prior to analysis, a region of interest (ROI) was chosen in every set of images. The ROI was typically selected based on minimal aggregates/clusters in the image to avoid skewing the results (clusters analysis was also performed to further avoid skewing the results). The ROIs were analyzed using multiple ParticleTracker add-on for ImageJ (see, e.g., information on https://sbalzarini-lab.org//ParticleTracker/index.html; Sbalzarini and Koumoutsakos, Journal of structural biology, 151(2):182-195 (2005)). Clusters are particle aggregates containing at least four NPs. For cluster analysis, ROI was typically not used, instead the full images were analyzed. Full images were first contrast enhanced, smooth, sharpened, and/or made binary for cluster analysis.

### Full Image Analysis Method:

Sample preparation was the same as steps (1) and (2) described above. In step (3), the imaging sample was prepared as described above; however, for measurement, the spectral settings of the confocal microscope were adjusted to account for the fluorescent particle's wavelength and intensity for detection. Images were taken at a z-stack height well above the aggregative bottom layer of the slide to avoid noise detection and monodispersed fluorescent particles were selected for imaging. In step (4), all ten (10) confocal images are read in with their original dimensions (generally 512×512 pixels) without selecting regions of interest. The entirety of the 10 images were read in as one file, before accumulating the diffusion coefficients. The data field selection for the particles was specific to the radius of the fluorescent particles to avoid false positives. The micron to pixel ratio was set to the actual scanning pixel ratio and provided a scale for the detection of the particles per pixel.

Using the full image method for diffusion coefficient analysis, a distribution curve of particles' diffusion coefficients was obtained. A geometric mean value of the distribution curve represents the diffusion coefficient of the entire population of particles being analyzed.

### Malvern Zetasizer Method

To measure particle diffusion in water, the samples were prepared by suspending dry nanospheres in DD water to achieve optimal concentration of 0.3-0.6 mg/ml. The samples were examined using Malvern Zetasizer Nanoseries. All size measurements were carried out at room temperature, using water parameters of viscosity 0.8872 cP, Dielectric constant of 78.50 and Refractive Index of 1.330 for DD water. Appropriate refractive index for each polymer known from literature was used. The scattered angle of 173° was used. Prior to each run the sample was vortexed and sonicated.

Upon analysis, the hydrodynamic diameter and diffusion were correlated using the Stokes-Einstein equation: D

$$D = \frac{{k}_{B} T}{6 π η r}$$

These data are provided in Table 1, column entitled “Dm in Water (cm2/s)* control particles”.

### 2. RESULTS

Coating was found to affect the surface charge and size of the polymeric cores (also referred to as “control particles” or “control NPs”) (FIG. 1). Most of the tested polymeric cores exhibited an increase in their hydrodynamic diameter when placed in 0.1% mucin solution compared to DIW (FIG. 1). Since none of these polymers swell in aquatic solutions, the increase in diameter demonstrates that mucin interacts with these polymeric cores, forming a coating. PBMAD core has shown the greatest increase (3-fold) while P(FA:SA) 20:80 core and PEG-PLGA (50:50) core, do not show any change. For PEG-PLGA core, this can be attributed to PEG residing on the surface, thus creating an interface that prevents mucin interactions. For P(FA:SA) 20:80 core, it can be attributed to its rapid hydrolysis in water.

The zeta potential of P(FA:SA) 20:80 core changed significantly (−53 to −8 mV), demonstrating the interactions of these polymeric cores with mucins. All tested polymeric cores have negative zeta potential in DIW, from −17 to −53 mV. When studied in 0.1% mucin solution, the zeta potentials significantly reduced to −16 to −7.7 mV (neat 0.1% mucin zeta potential was about −7.3 mV). PS and P(FA:SA) 20:80 cores showed the lowest zeta potential when exposed to mucins. The relatively high negative charge in DIW of PLA8 core (−48 mV) is attributed to its low molecular weight (MW), resulting in a higher percentage of acid end groups exposed on its surface. This may also explain why PLA8 core possesses relatively high bioadhesive force due to carboxylic end groups, resembling PBMAD and P(FA:SA) cores with their acidic side groups. This phenomenon was also observed for positively charged polymeric cores, demonstrating mucin's coating affects positively and negatively charged polymeric cores. Coating is attributed to mucin's glycoprotein's various molecular interactions such as disulfide bridging and electrostatic forces, that create a flexible array of alternating hydrophilic/hydrophobic regions. This explains why it can interact with various types of polymeric cores.

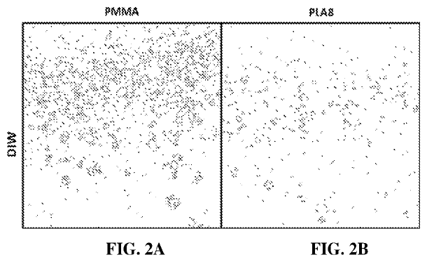

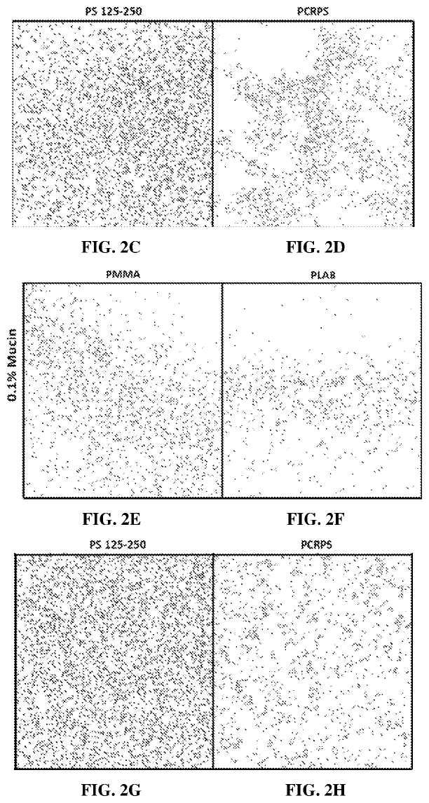

Next, the effect of mucins coating on polymeric cores aggregation was assessed as it affects particles' mobility, fate, persistence, toxicity and cellular uptake. Cluster formation in DIW and 0.1% mucins was analyzed (clusters are defined as aggregates with >4 particles, FIGS. 2A-2H). The results show that mucins coating has affected the extent of particles' aggregation. For example, PLA8 particle first exposed to 0.1% mucins has exhibited a 20% decrease in clusters formation when placed in 5% mucin solution compared to suspending it first in DIW. On the other hand, PCRPS particle showed an increase of 20% in clusters formation in 5% mucin when first coated with mucins. Cluster formation of PS 125-250 and PMMA particle was not been affected by coating. These may be because that coating reduces the polymeric cores' effective charge that is known to affect the stability of suspensions. More clusters also mean that a greater fraction of the particles diffuse effectively as bigger particles, thereby decrease their diffusion rate and may also impair their mucin penetration. On the other hand, it may decrease the burst release (which is a major limitation for drug delivery vehicles) by hindering the release of the drug.

The effect of mucin coating on the mucosal diffusion coefficient was evaluated using the selected region of interest method described above (Table 1). 540 nm polychromatic red PS (PCRPS) and PS 125-250 kDa (hydrophobic interactions), PLA8 (electrostatic interactions), and PMMA (mild to no interactions) cores were initially dispersed in 0.1% or DIW to form a coating, and then the effective diffusion of each of these coated particles in 5% mucus was tested.

TABLE 1
Average diffusion coefficients for control particles (“polymeric cores”) and
coated particles of PMMA 75 kDa, PS 125-250 kDa, and PLA 8 kDa.

| | | Dm in 5% | Dm in 5% | Enhancement | Enhancement |
|---|---|---|---|---|---|
| | Dm in Water | mucin | mucin | compared to | compared to |
| | (cm²/s)* | (cm²/s)** | (cm²/s)** | water (fold) | water (fold) |
| | control | control | coated | control | coated |
| Polymer | particles | particles | particles | particles | particles |
| PMMA | 1.38786E−10 | 1.90E−10 | 1.80E−10 | 1.37 | 1.30 |
| 75 kDa | | | | | |
| PS 125- | 1.27865E−10 | 2.20E−08 | 1.10E−07 | 172.06 | 860.28 |
| 250 kDa | | | | | |
| PLA | 9.2681E−11 | 3.40E−08 | 7.30E−07 | 366.85 | 7876.48 |
| 8 kDa | | | | | |

*Average diffusion coefficients for the control particles in water was determined from the Stokes-Einstein equation, using the hydrodynamic diameters from FIG. 6A.
**Average diffusion coefficients of coated and control particles in 5% mucin were determined from the Select Region of Interest confocal method described in the previous section.

As shown in Table, 1, control particles, i.e. PLA 8 kDa and PS 125-250 kDa cores with no coating, can be coated in the mucin solution, which showed an increased diffusion coefficient in 5% mucin compared to that in water. Prior to this study, particles were regarded being entrapped by mucin and show little to no diffusion. In contrast, the data shows that a biocoating of mucin increases particle diffusion in mucin.

Further, based on the Stokes-Einstein equation, particles would be expected to diffuse faster in water than mucin.

Stokes-Einstein equation:

$$D m = \frac{{k}_{B} T}{6 π η r}$$

where, Dₘ is the average diffusion coefficient, k_{B} is Boltzmann's constant, T is temperature, η is dynamic viscosity, and r is the radius of the spherical particle.

The addition of mucin to an aqueous solution is expected to result in increased viscosity and increased particle size. The increased viscosity (η) and the increased radius of the particles (r) should result in a decrease in the diffusion coefficient. However, the data shows that coated particles have an increased diffusion coefficient in mucin solution as compared to the diffusion coefficient in water, which is against the prediction from the Stokes-Einstein equation.

Additionally, the coated particles show an enhanced diffusion coefficient in mucin solution compared to the control particles (which does not contain a coating prior to addition into the mucin solution and was biocoated in vivo in the mucin solution) in the same mucin solution.

The effect of mucin coating on the mucosal diffusion coefficient was also evaluated using the full image method described above (FIGS. 3 and 4). As shown in FIGS. 3 and 4, polystyrene particles of 0.5 μm and 1 μm size have an increased diffusion coefficient in mucin compared to their diffusion coefficient in water. In FIGS. 3 and 4, the “PS control” refers to control particles measured in DI water; “control in mucin” refers to control particles (which were not coated prior to addition in mucin) measured in 5% mucin; and “PS 0.1 Coat in Mucin” and “PS 1.0 Coat in Mucin” refer to coated particles measured in 5% mucin, where “0.1” and “1” refer to the concentration of mucin solution used to coat the PS control particles.

### 3. CONCLUSIONS

The diffusion coefficient results of coated particles and control particles demonstrated that the coating particles with mucin can increase their Dₘ and reduce particles' aggregation, and will ultimately improve their oral uptake potency in vivo.

### REFERENCES

Sarosiek, et al. Biochem. Biophys. Res. Commun. 118, 523-531 (1984).
Witas, et al. Carbohydr. Res. 120, 67-76 (1983).
Lichtenberger Annu. Rev. Physiol. 57, 565-583 (1995).
Cu & Saltzman, Adv. Drug Deliv. Rev. 61, 101-114 (2009).
Jung, et al. European Journal of Pharmaceutics and Biopharmaceutics 50, 147-160 (2000).
Ensign, et al. Adv. Drug Deliv. Rev. 64, 557-570 (2012).
Kumari, et al. Colloids and Surfaces B: Biointerfaces 75, 1-18 (2010).
Panyam & Labhasetwar Adv. Drug Deliv. Rev. 55, 329-347 (2003).
Plapied, et al. Curr. Opin. Colloid Interface Sci. 16, 228-237 (2011).
Reineke, et al. J. Controlled Release 170, 477-484 (2013).
Reineke, et al. Proc. Natl. Acad. Sci. 110, 13803-13808 (2013).
Amidon Pharm. Res. 12, 413-420 (1995).
Bhavsar, et al. J. Controlled Release 110, 422-430 (2006).
Lawrence, et al. Int. J. Pharm. 140, 111-118 (1996).
Lawrence, et al. Adv. Drug Deliv. Rev. 19, 359-376 (1996).
Pillay & Fassihi J. Controlled Release 59, 229-242 (1999).
Minekus, et al. Alternatives to laboratory animals: ATLA (1995).
Lai, et al. Proc. Natl. Acad. Sci. 104, 1482-1487 (2007).
des Rieux, et al. J. Controlled Release 116, 1-27 (2006).
Mathiowitz, et al. Nature 386, 410 (1997).
Thanos, et al. J. Pharm. Sci. 92, 1677-1689 (2003).
Atuma, et al. Am. J. Physiol. Gastrointest. Liver Physiol. 280, G922-G929 (2001).
Eldridge, et al. J. Controlled Release 11, 205-214 (1990).
LeFevre Experientia 34, 120-122 (1978).
Shakweh, et al. European Journal of Pharmaceutics and Biopharmaceutics 61, 1-13 (2005).
Johansson, et al. Proc. Natl. Acad. Sci. 108 Suppl 1, 4659-4665 (2011).
Azagury, et al. Adv. Drug Deliv. Rev. 72, 127-143 (2014).
Khoo, et al. Biomaterials 34, 1281-1288 (2013).
Galey, et al. J. Invest. Dermatol. 67, 713-717 (1976).
Azagury, et al. J. Controlled Release 200, 35-41 (2015).
Arora & Garg Drug Discov. Today 7, 967-975 (2002).
Maisel, et al. J. Controlled Release 197, 48-57 (2015).
Cone Adv. Drug Deliv. Rev. 61, 75-85 (2009).
Thanos, et al. J. Bioact. Compatible Polym. 19, 247-266 (2004).
Furtado, et al. Int. J. Pharm. 347, 149-155 (2008).
Chickering Iii, et al. J. Controlled Release 48, 35-46 (1997).
de Sousa, et al. Int. J. Pharm. 500, 236-244 (2016).
Kulkarni & Feng Pharm. Res. 30, 2512-2522 (2013).
Lieleg, et al. Biophys. J. 98, 1782-1789 (2010).
Liu, et al. J. Controlled Release 222, 67-77 (2016).
Beloqui, et al. Adv. Drug Deliv. Rev. 106, 242-255 (2016).
Norris & Sinko J. Appl. Polym. Sci. 63, 1481-1492 (1997).
Jani, et al. J. Pharm. Pharmacol. 41, 809-812 (1989).
Pekarek, et al. Nature 367, 258 (1994).
Inchaurraga, et al. Drug Deliv. Transl. Res. 10, 1601-1611 (2020).
Kas J. Microencapsul. 14, 689-711 (1997).
Harush-Frenkel, et al. Biomacromolecules 9, 435-443 (2008).
Abdulkarim, et al. European Journal of Pharmaceutics and Biopharmaceutics 97, 230-238 (2015).
Griffiths, et al. European Journal of Pharmaceutics and Biopharmaceutics 97, 218-222 (2015).
Yang, et al. PloS one 7, e51054 (2012).
Berrecoso, et al. Drug Delivery and Translational Research 1-21 (2020).
Santos, et al. Journal of Controlled Release 60, 11-22 (1999).
Suknuntha, et al. Drug Dev. Ind. Pharm. 37, 408-418 (2011).
Authimoolam & Dziubla Polymers 8, 71 (2016).
Kettler, et al. Environmental toxicology and chemistry 33, 481-492 (2014).
Salopek, et al. Rudarsko-geolosko-naftni zbornik 4, 147 (1992).
Huang & Brazel J. Controlled Release 73, 121-136 (2001).
Hasan, et al. Int. J. Pharm. 344, 53-61 (2007).
Siegel, et al. J. Controlled Release 8, 223-236 (1989).
Maisel, et al. Nanomedicine 11, 1337-1343 (2016).
Magarkar, et al. Scientific reports 4, 5005 (2014).
Lamson, et al. Nature Biomedical Engineering 4, 84-96 (2020).
Verma, et al. Acta biomaterialia 31, 288-300 (2016).
Tuleu, et al. Int. J. Pharm. 180, 123-131 (1999).
Yuen Int. J. Pharm. 395, 9-16 (2010).
Trbojević-Stanković, et al. Histol. Histopathol. (2010).
Varum, et al. Eur. J. Pharm. Sci. 40, 335-341 (2010).
Hossain, et al. Pharm. Res. 7, 1163-1166 (1990).
Davis, et al. J. Pharm. Pharmacol. 53, 33-39 (2001).
Kararli Biopharm. Drug Dispos. 16, 351-380 (1995).
Estrellas, et al. Colloids and Surfaces B: Biointerfaces 173, 454-469 (2019).
Laulicht, et al. Macromolecular bioscience 12, 1555-1565 (2012).
Mathiowitz, et al. Nature 386, 410 (1997).
WO1997/026864 by Jacob & Mathiowitz.
Mathiowitz, et al. J Appl. Polym. Sci. 35, 755-774 (1988).
Swinehart J. Chem. Educ. 39, 333 (1962).
Azagury, et al. J. Controlled Release 183, 105-113 (2014).
Moser, et al. European journal of pharmaceutics and Biopharmaceutics 52, 103-112 (2001).
Rohman, et al. American Journal of Food Technology 7, 151-159 (2012).
Doolaanea, et al. Int. J. Pharm. Pharm. Sci 6, 228-232 (2014).
Reineke, et al. J. Controlled Release 170, 477-484 (2013).
Baker, et al. Nature protocols 9, 1771 (2014).
Gajjar, et al. Analyst 138, 3917-3926 (2013).
Sbalzarini & Koumoutsakos J. Struct. Biol. 151, 182-195 (2005).
Guillen, et al. Energy Fuels 6, 518-525 (1992).
Torabi, et al. Journal of Chromatography A 910, 19-30 (2001).
Anselmo & Mitragotri Adv. Drug Deliv. Rev. 108, 51-67 (2017).
Cui, et al. Adv Mater 25, 3468-3472 (2013).
Liu, et al. Soft Matter 8, 9235-9245 (2012).
Kettler, et al. Environmental toxicology and chemistry 33, 481-492 (2014).
Win & Feng Biomaterials 26, 2713-2722 (2005).
Guo, et al. Langmuir 30, 7206-7212 (2014).
Lai, et al. Proc. Natl. Acad. Sci. 104, 1482-1487 (2007).
Yi & Gao Nanoscale 9, 454-463 (2017).
Israelachvili Intermolecular and surface forces (Academic press, 2011).
Sotres, et al. Scientific reports 7, 7270 (2017).
Lock, et al. Scientific reports 8, 1-14 (2018).
Chickering Iii, et al. J. Controlled Release 48, 35-46 (1997).
Reineke, et al. Proc. Natl. Acad. Sci. 110, 13803-13808 (2013).
Furtado, et al. Int. J. Pharm. 347, 149-155 (2008).
Ye Int. J. Obes. 33, 606 (2009).

1. Coated particles comprising:
a core comprising a polymer or a hydrophobic drug; and
a coating comprising a glycoprotein or a combination of a sugar and a protein,
wherein the glycoprotein or the combination of sugar and protein surrounds the core.

2. The coated particles of claim 1, having an average hydrodynamic diameter in a range from 100 nm to 5000 nm, and optionally in a range from 100 nm to 2000 nm.

3. The coated particles of claim 1, wherein the core comprises a hydrophobic drug.

4. The coated particles of claim 1, wherein the core comprises a polymer and the polymer is biodegradable and biocompatible.

5-6. (canceled)

7. The coated particles of claim 4, wherein the polymer is selected from the group consisting of polystyrene, hydrogels, such as fibrin, collagen, gelatin, hyaluronic acid, alginate, cellulose, dextran, and agarose, poly(alkylene glycol), such as poly(ethylene glycol), poly(vinyl alcohol), poly(acrylic acid), polyacrylate, such as poly(methyl methacrylate), poly(2-hydroxyethyl methacrylate), poly(ethyleneglycolmethacrylate), poly(oligoethylene glycol methacrylate), poly(ethyleneglycol dimethacrylate), and poly(diacrylate), polyacrylamide, such as poly(isopropylacrylamide), poly(vinyl pyrrolidone), hydrophobic peptides, polyesters, such as poly(caprolactone), poly(hydroxyacids), such as poly(lactic acid), poly(glycolic acid), and poly(lactic acid-co-glycolic acid), polyhydroxyalkanoates, such as poly(3-hydroxybutyrate) and poly(4-hydroxybutyrate), polyanhydrides, such as poly(fumaric-co-sebacic acid), poly[butadiene-co-(maleic anhydride)], polysebacic acid, polyfumaric acid, poly(orthoesters), hydrophobic polypeptides, hydrophobic polyethers, such as poly(propylene oxide), poly(phosphazenes), polyesteramides, poly(alkylene alkylates), polyether esters, polyacetals, polycyanoacrylates, polyketals, polyhydroxyvalerates, polyalkylene oxalates, polyalkylene succinates, mixtures, and copolymers thereof.

8. The coated particles of claim 4, wherein the polymer is selected from the group consisting of polystyrene, poly(methyl methacrylate), poly(ethylene glycol), poly(lactic acid), poly[butadiene-co-(maleic anhydride)], poly(fumaric-co-sebacic acid), poly(glycolic acid), poly(lactic acid-co-glycolic acid), polysebacic acid, polyfumaric acid, mixtures, and copolymers thereof, optionally wherein the polymer is poly(lactic acid) or poly(lactic acid-co-glycolic acid), or a combination thereof.

9. (canceled)

10. The coated particles of claim 4, wherein the polymer is bioadhesive, optionally wherein the polymer has a bioadhesion force of about 100 mN/cm² or greater, or about 500 mN/cm² or greater: or wherein the hydrophobic drug is a Biopharmaceutical Classification System (“BCS”) Class IV drug.

11. (canceled)

12. The coated particles of claim 1, wherein the coating comprises a glycoprotein selected from the group consisting of collagen, mucins, transferrin, ceruloplasmin, immunoglobulins, histocompatibility antigens, human chorionic gonadotropin, thyroid-stimulating hormone, enzymes, such as alkaline phosphatase and patatin, proteins involved in cell-cell, virus-cell, bacterium-cell, and/or hormone-cell interactions, plasma proteins of coldwater fish, selectins, antibodies that interact with carbohydrates, proteins involved in hormone and drug actions, calnexin, calreticulin, notch and its analogs, proteins involved in the regulation of development, glycoproteins on the surface membranes of platelets.

13. The coated particles of claim 1, wherein the coating comprises a combination of (i) a sugar, an oligosaccharide, and/or a polysaccharide, and (ii) a protein, and wherein the sugar, oligosaccharide, and/or polysaccharide is covalently or non-covalently bound to the protein.

14. The coated particles of claim 13, wherein the sugar, oligosaccharide, and/or polysaccharide is/are selected from the group consisting of sucrose, glucose, fucose, mannose, galactose, xylose, an oligosaccharide thereof, and a polysaccharide thereof, and wherein the protein contains an amino acid bone comprising at least 50 repeated units of serine and threonine.

15. The coated particles of claim 1, wherein the coating further comprises one or more excipients and/or one or more active agents that are different from the hydrophobic drug.

16-17. (canceled)

18. The coated particles of claim 15, wherein the coating further comprises one or more active agents that are different from the hydrophobic drug, and wherein the one or more active agents are selected from the group consisting of small molecules, proteins, polypeptides, peptides, carbohydrates, nucleic acids, glycoproteins, lipids, antibodies/antigens, and combinations thereof, and
wherein when the active agent is or includes a protein and/or a glycoprotein, the protein or glycoprotein in the active agent is different from the protein or glycoprotein that forms the coating.

19. The coated particles of claim 1, wherein the core comprises a hydrophobic drug, and wherein the weight percentage of the hydrophobic drug in the coated particles is in a range from 0.01% to 10%, from 0.05% to 10%, from 0.1% to 10%, from 0.01% to 5%, from 0.05% to 5%, from 0.1% to 5% from 0.01% to 2%, from 0.05% to 2%, from 0.1% to 2%, from 0.01% to 1%, from 0.05% to 1%, or from 0.1% to 1%.

20. The coated particles of claim 15, wherein the weight percentage of the one or more active agents in the coated particles is in a range from 0.01% to 10%, from 0.05% to 10%, from 0.1% to 10%, from 0.01% to 5%, from 0.05% to 5%, from 0.1% to 5%, from 0.01% to 2%, from 0.05% to 2%, from 0.1% to 2%, from 0.01% to 1%, from 0.05% to 1%, or from 0.1% to 1%.

21. The coated particles of claim 1, wherein the coated particles have a diffusion coefficient in a mucin solution (3 to 7% wt) that is at least 1.1-fold, at least 1.3-fold, at least 2-fold, at least 5-fold, in a range from 1.3-fold to 30-fold, from 1.3-fold to 25-fold, from 2-fold to 30-fold, from 2-fold to 25-fold, from 5-fold to 30-fold, or from 5-fold to 25-fold greater than the diffusion coefficient of control particles consisting of the core in the same mucin solution, under the same measurement conditions; and/or
wherein the coated particles have a diffusion coefficient in a mucin solution (3 to 7% wt) that is least 1.1-fold, at least 1.3-fold, at least 2-fold, at least 5-fold, at least 10-fold, at least 20-fold, at least 30-fold, at least 40-fold, at least 50-fold, at least 100-fold, at least 150-fold, at least 160-fold, at least 170-fold, at least 200-fold, at least 250-fold, at least 300-fold, at least 500-fold, at least 1000-fold, at least 5000-fold, in a range from 1.3-fold to 10,000-fold, from 1.3-fold to 8000-fold, from 1.3-fold to 5000-fold, from 1.3-fold to 1000-fold, from 1.3-fold to 500-fold, from 1.3-fold to 200-fold, from 1.3-fold to 100-fold, from 1.3-fold to 30-fold, from 1.3-fold to 25-fold, from 2-fold to 30-fold, from 2-fold to 25-fold, from 5-fold to 30-fold, from 5-fold to 25-fold, from 100-fold to 10,000-fold, from 100-fold to 8000-fold, from 100-fold to 5000-fold, from 100-fold to 1000-fold, from 100-fold to 500-fold, from 150-fold to 10,000-fold, from 150-fold to 8000-fold, from 150-fold to 5000-fold, from 150-fold to 1000-fold, from 150-fold to 500-fold, or from 500-fold to 10,000-fold, greater than the diffusion coefficient of control particles consisting of the core only in water, under the same measurement conditions.

22. (canceled)

23. The coated particles of claim 1, wherein the diffusion coefficient of the coated particles in a mucin solution (3 to 7% wt) has a first enhancement factor compared to the diffusion coefficient of control particles in water, under the same measurement conditions, wherein the diffusion coefficient of the control particles in the same mucin solution has a second enhancement factor compared to the diffusion coefficient of the control particles in water, under the same measurement conditions, and wherein the first enhancement factor is at least 1.1-fold, at least 1.5-fold, at least 2-fold, at least 3-fold, at least 5-fold, at least 6-fold, at least 7-fold, at least 8-fold, at least 10-fold, at least 12-fold, at least 15-fold, at least 20 fold, in a range from 1.1-fold to 100-fold, from 1.1-fold to 80-fold, from 1.1-fold to 60-fold, from 1.1-fold to 50-fold, from 1.1-fold to 40-fold, from 1.1-fold to 30-fold, from 2-fold to 100-fold, from 2-fold to 80-fold, from 2-fold to 60-fold, from 2-fold to 50-fold, from 2-fold to 40-fold, from 2-fold to 30-fold, from 5-fold to 100-fold, from 5-fold to 80-fold, from 5-fold to 60-fold, from 5-fold to 50-fold, from 5-fold to 40-fold, or from 5-fold to 30-fold, such as about 1.1-fold, about 5-fold, or about 20-fold, greater than the second enhancement factor.

24. The coated particles of claim 1, showing a reduced number of clusters detected in a mucin solution (3 to 7% wt) by at least 5%, at least 10%, in a range from 5% to 50%, from 5% to 40%, from 10% to 50%, or from 20% to 50%, compared to the number of clusters detected for control particles in the same mucin solution, under the same measurement conditions; and/or
wherein when the coated particles are administered to a mammal, the systemic uptake is in a range from 10% to 90%, from 15% to 85%, from 15% to 80%, from 15% to 70%, from 15% to 60%, from 15% to 50%, from 20% to 90%, from 20% to 80%, from 20% to 70%, from 20% to 60%, or from 20% to 50% in a mammal, as measured using Fourier Transform Infrared spectroscopy.

25. (canceled)

26. The coated particles of claim 1, wherein the core and the coating are not or do not contain polyethylene glycol; and/or wherein the coating is not or does not contain lectins.

27. A pharmaceutical formulation comprising the coated particles of claim 1, and a pharmaceutically acceptable carrier and/or excipient, optionally wherein the pharmaceutical formulation is in a form suitable for oral administration or intravaginal administration.

28. (canceled)

29. A method for treating a disease or disorder in a subject in need thereof comprising:
(i) administering to the subject the pharmaceutical formulation of claim 27, wherein step (i) occurs one or more times.

30-31. (canceled)
